SEQUENCE LISTING (48 sequences)

SEQ ID NO: 1 - DNA, 138 bp, Sus scrofa
  source 1..138: mol_type = genomic DNA
tcctgtgctgtggcctcctgaggagcgatgacggagtataagctggtggtggtgggcgctggaggtgtggggaagagtgccctgaccatccagcttatccagaaccactttgtggatgagtacgaccccaccatagag

SEQ ID NO: 2 - DNA, 138 bp, synthetic construct
  source 1..138: mol_type = other DNA
tcctgtgctgtggcctcctgaggagcgatgacggagtataagctggtggtggtgggcgctgtaggtgtggtgaagagtgccctgaccatccagcttatccagaaccactttgtggatgagtacgaccccaccatagag

SEQ ID NO: 3 - DNA, 138 bp, synthetic construct
  source 1..138: mol_type = other DNA
tcctgtgctgtggcctcctgaggagcgatgacggagtataagctggtggtggtgggcgctgtaggtgtgttgaagagtgccctgaccatccagcttatccagaaccactttgtggatgagtacgaccccaccatagag

SEQ ID NO: 4 - DNA, 143 bp, Sus scrofa
  source 1..143: mol_type = genomic DNA
aaacagctgtaatacctattaatggttcacctcgaacaccaaggcgaggtcagaacaggagtgcacggatagcaaaacaactagaaaatgatacaagaattattgaagttctctgtaaagaacatgaatgtaatatagatgag

SEQ ID NO: 5 - DNA, 141 bp, synthetic construct
  source 1..141: mol_type = other DNA
aaacagctgtaatacctattaatggttcacctcgaacaccaacgaggtcagaacaggagtgcacggatagcaaaacaactagaaaatgatacaagaattattgaagttctctgtaaagaacatgaatgtaatatagatgag

SEQ ID NO: 6 - DNA, 141 bp, synthetic construct
  source 1..141: mol_type = other DNA
aaacagctgtaatacctattaatggttcacctcgaacaccaaggaggtcagaacaggagtgcacggatagcaaaacaactagaaaatgatacaagaattattgaagttctctgtaaagaacatgaatgtaatatagatgag

SEQ ID NO: 7 - DNA, 85 bp, Sus scrofa
  source 1..85: mol_type = genomic DNA
atatttatccaaacattattgctatggggtttcctgcagaaagacttgaaggcgtatacaggaacaatattgatgatgtagtaag

SEQ ID NO: 8 - DNA, 86 bp, synthetic construct
  source 1..86: mol_type = other DNA
atatttatccaaacattattgctatggggtttcctgcagaaagacttgaaggcgtaatacaggaacaatattgatgatgtagtaag

SEQ ID NO: 9 - DNA, 83 bp, synthetic construct
  source 1..83: mol_type = other DNA
atatttatccaaacattattgctatggggtttcctgcagaaagacttgaaggcgtacaggaacaatattgatgatgtagtaag

SEQ ID NO: 10 - DNA, 187 bp, Sus scrofa
  source 1..187: mol_type = genomic DNA
tactcccctgccctcaataagctgttttgccagctggccaagacctgcccggtgcagctgtgggtcagctcgccacccccgcctggcacccgtgtccgcgccatggccatctacaagaagtcagagtacatgaccgaggtggtgaggcgctgtccccaccatgagcgcagctctgactatagcgatg

SEQ ID NO: 11 - DNA, 188 bp, synthetic construct
  source 1..188: mol_type = other DNA
tactcccctgccctcaataagctgttttgccagctggccaagacctgcccggtgcagctgtgggtcagctcgccacccccgccttggcacccgtgtccgcgccatggccatctacaagaagtcagagtacatgaccgaggtggtgaggcgctgtccccaccatgagcgcagctctgactatagcgatg

SEQ ID NO: 12 - DNA, 186 bp, synthetic construct
  source 1..186: mol_type = other DNA
tactcccctgccctcaataagctgttttgccagctggccaagacctgcccggtgcagctgtgggtcagctcgccacccccgccggcacccgtgtccgcgccatggccatctacaagaagtcagagtacatgaccgaggtggtgaggcgctgtccccaccatgagcgcagctctgactatagcgatg

SEQ ID NO: 13 - DNA, 138 bp, Sus scrofa; identical to SEQ ID NO: 1
  source 1..138: mol_type = genomic DNA

SEQ ID NO: 14 - DNA, 139 bp, synthetic construct
  source 1..139: mol_type = other DNA
tcctgtgctgtggcctcctgaggagcgatgacggagtataagctggtggtggtgggcgctggaggttgtggggaagagtgccctgaccatccagcttatccagaaccactttgtggatgagtacgaccccaccatagag

SEQ ID NO: 15 - DNA, 20 bp, synthetic construct
  source 1..20: mol_type = other DNA
ctcctgttctgacctcgcct

SEQ ID NO: 16 - DNA, 20 bp, synthetic construct
  source 1..20: mol_type = other DNA
cgcggacacgggtgccaggc

SEQ ID NO: 17 - DNA, 20 bp, synthetic construct
  source 1..20: mol_type = other DNA
tggtgggcgctggaggtgtg

SEQ ID NO: 18 - DNA, 20 bp, synthetic construct
  source 1..20: mol_type = other DNA
aaagacttgaaggcgtatac

SEQ ID NO: 19 - DNA, 200 bp, synthetic construct
  source 1..200: mol_type = other DNA
atggtctggccacgctgaatgctgcttcctccactgcagtcctgtgctgtggcctcctgaggagcgatgacggagtataagctggtggtggtgggcgctgtaggtgtggtgaagagtgccctgaccatccagcttatccagaaccactttgtggatgagtacgaccccaccatagaggtgagcctccagcccgcatcccg

SEQ ID NO: 20 - DNA, 20 bp, synthetic construct
  source 1..20: mol_type = other DNA
agttgggggcagaagagaat

SEQ ID NO: 21 - DNA, 20 bp, synthetic construct
  source 1..20: mol_type = other DNA
gattccagagtgagggtgct

SEQ ID NO: 22 - DNA, 20 bp, synthetic construct
  source 1..20: mol_type = other DNA
cttggcctctcatccttccc

SEQ ID NO: 23 - DNA, 20 bp, synthetic construct
  source 1..20: mol_type = other DNA
ccacgctgtgtcgaaaagtg

SEQ ID NO: 24 - DNA, 20 bp, synthetic construct
  source 1..20: mol_type = other DNA
cttctaattcgggtgcgtgc

SEQ ID NO: 25 - DNA, 20 bp, synthetic construct
  source 1..20: mol_type = other DNA
aatgaccacttgcttccggt

SEQ ID NO: 26 - DNA, 20 bp, synthetic construct
  source 1..20: mol_type = other DNA
gggggtcgttgtctttacct

SEQ ID NO: 27 - DNA, 22 bp, synthetic construct
  source 1..22: mol_type = other DNA
aagctaacagccaactgtgaaa

SEQ ID NO: 28 - RNA, 20 nt, synthetic construct
  source 1..20: mol_type = other RNA
cgcggacacgggtgccaggc

SEQ ID NO: 29 - RNA, 20 nt, synthetic construct
  source 1..20: mol_type = other RNA
ctcctgttctgacctcgcct

SEQ ID NO: 30 - RNA, 20 nt, synthetic construct
  source 1..20: mol_type = other RNA
tggtgggcgctggaggtgtg

SEQ ID NO: 31 - RNA, 20 nt, synthetic construct
  source 1..20: mol_type = other RNA
cgtctagggttcctgcaatc

SEQ ID NO: 32 - RNA, 20 nt, synthetic construct
  source 1..20: mol_type = other RNA
acttggctgttccggattgc

SEQ ID NO: 33 - RNA, 20 nt, synthetic construct
  source 1..20: mol_type = other RNA
agtcgacccacagctgcact

SEQ ID NO: 34 - RNA, 20 nt, synthetic construct
  source 1..20: mol_type = other RNA
tatgggtctttgagcaatgc

SEQ ID NO: 35 - RNA, 20 nt, synthetic construct
  source 1..20: mol_type = other RNA
caccaaggcgaggtcagaac

SEQ ID NO: 36 - RNA, 20 nt, synthetic construct
  source 1..20: mol_type = other RNA
tctagatgcaagattatttc

SEQ ID NO: 37 - RNA, 20 nt, synthetic construct
  source 1..20: mol_type = other RNA
ggcgctcttccccacgccac

SEQ ID NO: 38 - RNA, 20 nt, synthetic construct
  source 1..20: mol_type = other RNA
ctcgtggtggtgggcgccgg

SEQ ID NO: 39 - RNA, 20 nt, synthetic construct
  source 1..20: mol_type = other RNA
ggtggtgggcgccggtggcg

SEQ ID NO: 40 - RNA, 20 nt, synthetic construct
  source 1..20: mol_type = other RNA
ctcaccatccttacactgga

SEQ ID NO: 41 - RNA, 20 nt, synthetic construct
  source 1..20: mol_type = other RNA
tatggttccttgagcatagc

SEQ ID NO: 42 - RNA, 20 nt, synthetic construct
  source 1..20: mol_type = other RNA
ccccacgaagaggtcagaac

SEQ ID NO: 43 - RNA, 20 nt, synthetic construct
  source 1..20: mol_type = other RNA
tatagatgcaagattatttc

SEQ ID NO: 44 - DNA, 200 bp, Sus scrofa
  source 1..200: mol_type = genomic DNA
atggtctggccacgctgaatgctgcttcctccactgcagtcctgtgctgtggcctcctgaggagcgatgacggagtataagctggtggtggtgggcgctgtaggtgtggggaagagtgccctgaccatccagcttatccagaaccactttgtggatgagtacgaccccaccatagaggtgagcctccagcccgcatcccg

SEQ ID NO: 45 - DNA, 200 bp, Bos sp.
  source 1..200: mol_type = genomic DNA
ggctccctcggcaggtgggaccggcgaccactgcagcgcctcgtgctgcggtctcttgaggagcaatgacggagtataagctcgtggtggtgggcgccgttggcgtggggaagagcgccctgactatccagctcattcagaatcacttcgtggacgagtacgaccccaccatcgaggtgagcccgcctggctcctgtccg

SEQ ID NO: 46 - protein, 189 aa, Sus scrofa
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKVRKLSPPDEGGPGCLSCKCLLS

SEQ ID NO: 47 - protein, 189 aa, Sus scrofa [sequence omitted]

SEQ ID NO: 48 - protein, 189 aa, Sus scrofa
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

(19) United States
(12) Patent Application Publication
(10) Pub. No.: US 2026/0152722 A1
(43) Pub. Date: Jun. 4, 2026

(54) GENETICALLY MODIFIED CELLS

(71) Applicant: IVY FARM TECHNOLOGIES LIMITED, Oxford (GB)

(72) Inventors: Florian HAHN, Oxford (GB); Rowan RIMINGTON, Oxford (GB); James SHELFORD, Oxford (GB); Caitlin DORAN, Oxford (GB); Lucy WILKINSON, Oxford (GB); Alasdair RUSSELL, Cambridge (GB); Michael GILL, Cambridge (GB); Amy SMITH, Cambridge (GB)

(21) Appl. No.: 19/113,653

(22) PCT Filed: Sep. 29, 2023
(86) PCT No.: PCT/GB2023/052528

§ 371 Date: Mar. 20, 2025

(30) Foreign Application Priority Data
Sep. 30, 2022 (GB) 2214410.9

(51) Int. Cl. C12N 5/077

(52) U.S. Cl. CPC C12N 5/0658; C12N 2510/02; C12N 2510/04

(57) ABSTRACT

The invention relates to a modified animal cell and a method of producing the modified animal cell having a genetic modification in one or more genes associated with genome surveillance, cell cycle control and/or cell death control. The animal cell is selected from an animal species suitable for human or animal consumption. The animal cell generally has a genetic modification in the RB1, TP53 or RAS genes.

### STATEMENT OF PRIORITY

This application is a 35 U.S.C. § 371 national phase application of PCT Application No. PCT/GB2023/052528 filed Sep. 29, 2023, which claims the benefit of and priority to British Application No. 2214410.9 filed Sep. 30, 2022, the entire contents of each of which are incorporated by reference herein.

### STATEMENT REGARDING ELECTRONIC FILING OF A SEQUENCE LISTING

A Sequence Listing in XML format, submitted under 37 C.F.R. § 1.831-1.834, entitled 1553-28_ST26.xml, 45,861 bytes in size, generated on Sep. 28, 2023 and filed electronically, is provided in lieu of a paper copy. This Sequence Listing is hereby incorporated by reference into the specification for its disclosures.

### FIELD OF THE INVENTION

The present invention relates to immortalised primary non-human animal cells for cultivated meat for human or animal consumption and methods for producing such cells.

### BACKGROUND OF THE INVENTION

The world population is set to increase to almost 10 billion people within the next 50 years. As a result, there will be nearly two billion additional people to feed by 2050. The rising population coupled with a growing appetite for meat in many countries will lead to an increase in global demand for meat by approximately 73% by the year 2050. The agricultural industry will have to scale up production, potentially doubling in size, in order to meet this demand. 39% of the earth's habitable land is currently used to produce feed to rear livestock for the meat industry. It takes three years to rear a single cow for slaughter, or 6-12 months for pigs and poultry. Therefore, a large area of arable land is required to feed these animals to term. Currently, 80 billion animals are slaughtered each year for meat with 1.2 billion slaughtered in the UK alone.

Cultivated meat has the potential to address the substantial global problems associated with livestock farming and the environmental impact of meat production along with animal welfare, food security and human health. Cultivated meat is a meat produced by in vitro cell cultures of animal cells. It is a form of cellular agriculture, with such agricultural methods being explored in the context of increased consumer demand for protein. Cellular agriculture relates to the production of animal-sourced foods from cell culture.

Cultivated meat is produced using tissue engineering techniques traditionally used in regenerative medicines and requires cell lines, usually stem cells. Stem cells are undifferentiated cells which have the potential to become many or all of the required kinds of specialized cell types. While pluripotent stem cells are often thought of as the ideal starting cell, the most prominent example of this subcategory of stem cell are embryonic stem cells which due to ethical issues are controversial for use in research. As a result, induced pluripotent stem cells (iPSCs) have been developed. iPSCs are multipotent blood and skin cells that are artificially regressed to a pluripotent state enabling them to differentiate into a greater range of cells. The alternative to iPSCs involves the use of multipotent adult stem cells which give rise to muscle cell lineages or unipotent progenitors which can differentiate into muscle cells. Favourable characteristics of stem cells which make them suitable for cultivated meat production include immortality, increased proliferative ability, lack of reliance on adherence, serum independence and easy differentiation into tissue.

Stem cells used to generate cell lines can be collected from a primary source, i.e., through a biopsy on an animal under local anaesthesia and can also be established from secondary sources such as cryopreserved cultures. However, somatic cells isolated from tissues/organs often used in food consumption (e.g. muscle, fat, and fibroblasts) from agriculturally relevant species (e.g. pigs, cows, chickens) have a limited lifespan when grown in vitro. Although it is possible to isolate primary cell lines from pigs (myoblasts, myofibroblasts, fibroblasts, adipose derived stem cells and epithelial cells) the ability to propagate these cell lines with efficient doubling times and for long term is not feasible.

The culture medium is an essential component of in vitro cultivation and is responsible for providing the macromolecules, nutrients and growth factors necessary for cell proliferation. Sourcing growth factors is one of the barriers to an efficient process of producing cultivated meat. Traditionally, sourcing growth factors involves the use of foetal bovine serum (FBS). FBS is a blood product extracted from foetal cows. Besides the ethical consideration, FBS also ensures that cultivated meat is not totally independent of the use of animals. FBS is also the most costly constituent of the process used to produce cultivated meat, priced at around $1000 per litre. Additionally, the chemical composition of FBS varies greatly between each animal source, so it cannot be uniformly quantified chemically. FBS is utilised as it conveniently assists in mimicking the process of muscle development in vivo. Growth factors needed for tissue development are predominantly provided through an animal's bloodstream, and no single fluid other than FBS can single-handedly deliver all these components. The current alternative to FBS is to generate each growth factor individually using recombinant protein production. In this process, the genes coding for the specific factor are integrated into bacteria which are then fermented. However, due to the added complexity of this process, it is particularly expensive.

It has previously been shown that human fibroblasts can be immortalised by introducing the telomerase catalytic subunit (hTERT), a hyperactive mutant of the H-Ras gene (H-RasG12V) and the large T antigen from simian virus 40 (SV40 LT), which binds and inactivates the cellular proteins p53 (encoded by TP53) and pRB (encoded by RB1) to uncouple the cell cycle (Hahn et al, Nature volume 400, pages 464-468 (1999)). Others have confirmed that exogenous introduction of hTERT and a hyperactive H-Ras (or relative, e.g. N-Ras), combined in some cases with TP53 inactivation, can immortalise both human fibroblasts, and also human epithelial cells (Yang et al, Carcinogenesis, Volume 28, Issue 1, January 2007, Pages 174-182; Toouli et al Oncogene volume 21, pages 128-139 (2002)). In relation to cell types relevant to cellular agriculture (e.g. muscle progenitor cells), human myoblasts have been immortalised using a combination of exogenous hTERT and mutant cyclin dependent kinase 4 (CDK4) (Zhu et al, Aging cell, Volume 6, Issue 4, August 2007, pages 515-523). This work has been extended into agriculturally relevant species where porcine and bovine fibroblasts have been immortalised by introducing exogenous TERT, mutant CDK4, and cyclin D1 expression (Donai et al, J Biotechnol. 2014 Apr. 20; 176: pages 50-7).

A common theme to approaches for the immortalisation of cells to produce cell lines for cellular agriculture is the addition of exogenous TERT. Another commonality is the need to introduce foreign genes (e.g. CDK4, cyclin D1, SV40 LT) into cells to immortalise them.

There is a need to establish immortalised cell lines. The inventors have addressed this need by providing modified animal cells that comprise modification of endogenous genes and do not require the expression of exogenous nucleic acid constructs.

### SUMMARY OF THE INVENTION

In a first aspect, the invention relates to a modified non-human animal cell having a genetic modification in one or more genes associated with genome surveillance, cell cycle control and/or cell death control and wherein the animal is of an animal species suitable for human or animal consumption, optionally used in agriculture.

In one embodiment, the modification immortalises the cell.

In one embodiment, the animal is selected from a pig, bovine, poultry, sheep, goat, fish, crustaceans or mollusc.

In one embodiment, the modified cell is a somatic cell.

In one embodiment, the modified cell is selected from one of the following cell types: myoblast, fibroblast, myofibroblast, adipose derived stem cell, epithelial cell, mesenchymal stem cell, satellite cell or hepatocyte.

In one embodiment, said cell does not express an exogenous nucleic acid to manipulate the genome surveillance, cell cycle control and/or cell death control pathway.

In one embodiment, the animal cell has a genetic modification in one or more of the following genes: RB1, TP53, and/or a RAS gene.

In one embodiment, the animal cell has a genetic modification in RB1.

In one embodiment, the animal cell has a genetic modification in TP53.

In one embodiment, the animal cell has a genetic modification in a RAS gene.

In one embodiment, the animal cell has a genetic modification in RB1 and in TP53.

In one embodiment, the animal cell has a genetic modification in RB1 and in a RAS gene.

In one embodiment, the animal cell has a genetic modification in TP53 and in a RAS gene.

In one embodiment, the animal cell has a genetic modification in RB1, TP53 and in a RAS gene.

In one embodiment, the RAS gene is HRAS, NRAS, or KRAS.

In one embodiment, the RAS gene is HRAS.

In one embodiment, the modification is in the promoter region or coding region of the one of more genes, or is a regulatory element that regulates one or mor of the genes.

In one embodiment, the modification is introduced using targeted genome modification.

In one embodiment, an endonuclease is used.

In one embodiment, the endonuclease is selected from TALEN, ZFN or CRISPR/Cas9.

In one embodiment, wherein the gene is selected from one or both of RB1 and/or TP53 and the modification is a loss of function modification.

In one embodiment, the loss of function modification comprises a knock-out of the gene.

In one embodiment, the gene is a RAS gene and the modification is a hyperactivation modification, optionally, wherein the RAS gene is HRAS, NRAS, or KRAS.

In one embodiment, the hyperactivation modification comprises one or more amino acid substitutions.

In one embodiment, the one or more amino acid substitutions comprises substituting the glycine at positions 12 or 13 or the Glutamine at position 61 of SEQ ID NO: 46, 47, or 48.

In one embodiment, the one or more amino acid substitutions comprises substituting the glycine at positions 12 or 13 of SEQ ID NO: 46, 47, or 48, wherein the one or more amino acid is selected from a list comprising alanine, cysteine, aspartic acid, arginine, serine, and valine.

In one embodiment, the one or more amino acid substitutions comprises substituting the glycine at positions 12 or 13 of SEQ ID NO: 46, 47, or 48 with valine.

In one embodiment, the one or more amino acid substitutions comprises substituting the glycine at position 61 of SEQ ID NO: 46, 47, or 48, wherein the one or more amino acid is selected from a list comprising glutamic acid, histidine, lysine, proline, and arginine.

In one embodiment, the one or more amino acid substitutions comprises substituting the glycine at position 12 of SEQ ID NO: 1 with valine.

In a second aspect, the invention relates to a method of producing cultivated meat or a cultured meat product comprising culturing the modified animal cell as described herein.

In one embodiment, the method comprises continuous or batch culture of the modified cell.

In one embodiment, the method comprises the step of forming the cells into a tissue like structure.

In one embodiment, the cells are formed into a muscle tissue like structure.

In another aspect, the invention relates to a method of producing the modified animal cell as described herein, wherein the method comprises introducing a genetic modification in one or more genes associated with genome surveillance, cell cycle control and/or cell death control and wherein the animal is an animal suitable for human or animal consumption, optionally used in agriculture.

In another aspect, the invention relates to a method of immortalising an animal cell, wherein the method comprises introducing a genetic modification in one or more genes associated with genome surveillance, cell cycle control and/or cell death control and wherein the animal is an animal suitable for human or animal consumption, optionally used in agriculture.

In one embodiment, the genetic modification alters the expression or function of one or more genes associated with genome surveillance, cell cycle control and/or cell death control.

In one embodiment, the animal cell is immortalised.

In one embodiment, the animal is selected from a pig, bovine, poultry, sheep, goat, fish, crustaceans or mollusc.

In one embodiment, the modified cell is a somatic cell.

In one embodiment, the modified cell is selected from one of the following cell types: myoblast, fibroblast, myofibroblast, adipose derived stem cell, epithelial cell, mesenchymal stem cell, satellite cell or hepatocyte.

In one embodiment, the cell does not express an exogenous nucleic acid to manipulate the genome surveillance, cell cycle control and/or cell death control.

In one embodiment, the animal cell has a genetic modification in one or more of the following genes: RB1, TP53, and/or a RAS gene.

In one embodiment, the animal cell has a genetic modification in RB1.

In one embodiment, the animal cell has a genetic modification in TP53.

In one embodiment, the animal cell has a genetic modification in a RAS gene.

In one embodiment, the animal cell has a genetic modification in RB1 and in TP53.

In one embodiment, the animal cell has a genetic modification in RB1 and in a RAS gene.

In one embodiment, the animal cell has a genetic modification in TP53 and in a RAS gene.

In one embodiment, the animal cell has a genetic modification in RB1, TP53 and in a RAS gene.

In one embodiment, the RAS gene is HRAS, NRAS, or KRAS.

In one embodiment, the RAS gene is HRAS.

In one embodiment, the modification is made to the promoter region or coding region of the one of more genes.

In one embodiment, the modification is introduced using targeted genome modification.

In one embodiment, an endonuclease is used.

In one embodiment, the endonuclease is selected from TALEN, ZFN or CRISPR/Cas9.

In one embodiment, the gene is selected from one or both of RB1 and/or TP53 and the modification is a loss of function modification.

In one embodiment, the loss of function modification comprises a knock-out of the gene.

In one embodiment, the gene is a RAS gene and the modification is a hyperactivation modification, optionally, wherein the RAS gene is HRAS, NRAS, or KRAS.

In one embodiment, the hyperactivation modification comprises one or more amino acid substitutions.

In one embodiment, the one or more amino acid substitutions comprises substituting the glycine at positions 12 or 13 or the Glutamine at position 61 of SEQ ID NO: 46, 47, or 48.

In one embodiment, the one or more amino acid substitutions comprises substituting the glycine at positions 12 or 13 of SEQ ID NO: 46, 47, or 48, wherein the one or more amino acid is selected from a list comprising alanine, cysteine, aspartic acid, arginine, serine, and valine.

In one embodiment, the one or more amino acid substitutions comprises substituting the glycine at positions 12 or 13 of SEQ ID NO: 46, 47, or 48 with valine.

In one embodiment, the one or more amino acid substitutions comprises substituting the glycine at position 61 of SEQ ID NO: 46, 47, or 48, wherein the one or more amino acid is selected from a list comprising glutamic acid, histidine, lysine, proline, and arginine.

In one embodiment, the one or more amino acid substitutions comprises substituting the glycine at position 12 of SEQ ID NO: 1 with valine.

In one embodiment, the one or more amino acid substitutions comprises substituting the glycine at position 12 of SEQ ID NO: 1 with valine.

In another aspect, the invention relates to a cultivated or cultured animal tissue or a cultivated or cultured meat product comprising a modified cell as described herein.

In one embodiment, the culture is a suspension culture.

In another aspect, the invention relates to a use of the modified animal cell as described herein for cellular agriculture.

In another aspect, the invention relates to a method for producing an immortalised animal cell line comprising the method as described herein.

A guide RNA for use in a method of producing the modified, immortalised cell, or immortalised animal cell line as described herein.

In another aspect, the invention relates to a guide RNA comprising any sequence selected from SEQ ID NOs. 15, 16, 17, 18.

In one embodiment, the guide RNA is for use in a method of producing the modified or immortalised cell as described herein.

In one embodiment, the modified cell is a modified cell as described herein.

In another aspect, the invention relates to a kit of parts comprising the guide RNA as described herein.

### BRIEF DESCRIPTION OF THE FIGURES AND TABLES

The invention is further described in the following non-limiting figures and tables.

FIG. 1, panel A is a schematic representation of the key cell cycle regulatory programmes, highlighting the contribution of the key CRISPR target genes (RB1, TP53 and Ras) to the different phases of DNA replication along with the regulatory programmes targeted for CRISPR-Cas9 gene editing (pRB/E2F, p53 and Ras/MEK/ERK pathways). FIG. 1, panel B is a workflow of CRISPR-Cas9 editing. Single cell suspensions were incubated with Cas9 and sgRNA ribonucleoprotein complexes and then subjected to nucleofection (Amaxa 4D, Lonza). Successful editing was assessed by amplicon sequencing and trace deconvolution. Extension of replicative capacity was determined by assessing proliferation (cell doublings) and transcriptional changes (qPCR).

FIG. 2 is a table highlighting the CRISPR targets genes (single, double, and triple editing targets) in three isolated primary porcine cell lines, IF-p036-A, O and AD. The lines highlighted in red boxes are the tripled edited cell lines currently being evaluate for their doubling times and immortalisation status, relative non-edited controls.

FIG. 3 is two bar graphs that represent the relative efficiency of H-RasG12V, TP53 and RB1 editing in two primary porcine cell lines, IF-p036-A and IF-p036-O. % editing was calculated using ICE analysis (Inference of CRISPR Edits).

FIG. 4 is a bar graph that shows the efficient multiplex CRISPR editing of a primary porcine cell line. The bars represent the relative efficiency of H-RasG12V, TP53 and RB1 editing in the serum free cell line, IF-p036-AD. Percentage (%) editing was calculated using ICE analysis (Inference of CRISPR Edits).

FIG. 5 shows porcine muscle derived cell lines edited via CRISPR/cas9 for HrasG12V (−/+), TP53 (−/−) and RB1 (−/−) display a growth advantage, reduced dependence on extracellular matrices and growth factors when compared to cas9 controls. a) Light micrographs of IFp036-A-27-A and IFp036-O-31-A triple edit CRISPR cell lines and respective cas9 controls at P15 in cell culture. b) Cumulative generations and c) doubling times of parental (IFp036-A/O), CRISPR and cas9 cell lines. d) Doubling times of IFp036-A-27-A and cas9 cell lines following removal of the extracellular matrix Matrigel, evidencing retained growth in triple edited CRISPR lines compared to cas9 controls. e) Doubling times are reduced in both IFp036-A-27-A and cas9 cell lines following removal of bFGF, however, edited cell lines evidence a reduced dependence compared to cas9 controls. These doubling times are however significantly higher than when cultured with bFGF (d vs. inset of e). Data presented as mean+/−SD.

FIG. 6 is a graph that shows the percentage of porcine cells that displayed successful editing after being nucleofected with RNP across a range of electrical and buffer conditions. Successful editing was assessed via amplicon sequencing. The percentage of total editing and gene knock-out (KO) were assessed.

FIG. 7 is two graphs that show; panel A: the percentage editing of cells when highly efficient sgRNAs identified for RB1, TP53 and H-RAS genes were used in the gene editing process; and panel B: the gene knock-out efficiency (%) in porcine cells when simultaneous multiplex triple-gene knock-out (KO) was carried out is shown.

FIG. 8 is a graph that shows the stable gene silencing (KO) of TP53 and RB1 genes and Knock-In (KI) of hyperactive H-Ras in the IVYp036-A cell line. Primary porcine cells were nucleofected with RNP targeting H-Ras alone or in combination with single stranded DNA oligonucleotide donor (ssODN) sequences harbouring the G12V substitution. Two independent ssODNs and two different concentrations of Cas9 were assessed. Sanger sequencing of the targeted loci, followed by trace deconvolution is shown (box). Note that for cell lines names in this patent IVY and IF are interchangeable. Example IVYp036-A=IFp036-A.

FIGS. 9A-9B are two graphs that show Knock-In of H-RasG12V and KO of TP53 and RB1 in two primary porcine myoblast lines (IVYp036-A and -O). * indicates a sequencing failure.

FIG. 10 is two graphs that show triple-edited myoblasts show characteristics of immortalisation. Panel A: A graph showing the fold difference using quantitative RT-PCR demonstrating transcriptional changes, relative to a housekeeping gene, across a panel of cell cycle regulatory genes. Panel B: A graph that shows the cell proliferation of triple-edited and control cell lines with/without supplementation of basic fibroblast growth factor (FGF2).

FIG. 11 is two graphs that show the knockout (KO) score for TP53 (red dots) and RB (green dots) and knockin (KI) score for HRas (blue dots) in the 3 edited cell pools at days 3, 10 and 17 following CRISPR mediated gene-editing of the IF p044 A cell line (left panel) and IF p044 C cell line (right panel). For each gene at the indicated time points, the dots from left to right represent cell pool 1, 2 and 3.

FIG. 12. Editing efficiencies and growth data from porcine myoblast cell lines. Cells were edited using one sgRNA against P53 (a) and RB1 (b) respectively or both in combination (c). Editing efficiencies (knockout) were measured on two to three time points post editing. For a growth assay, cells were seeded into triplicate flasks (from growth period 2 onwards) and grown on Matrigel coated flasks (d) or on plastic (e) in adherence for 8 passages. Doubling times were compared to a control line (transfection with Cas9 only, no sgRNA; Ctrl). As a reference point, the 8-passage average doubling time of a P53^{-/-}/RB1^{-/-}/HRAS^{G12V/-} cell line is also plotted in graphs (d) and (e).

FIG. 13. Editing efficiencies and growth data from porcine adipose derived stem cells (ADSC) cell lines. Cells were edited using one sgRNA against P53 (a) and RB1 (b) respectively or both in combination (c) or as a triple edit together with an HRAS^{G12V} knockin (d). Editing efficiencies (knockout for P53 and RB1; knockin for HRAS) were measured on three time points post editing. For a growth assay, cells were seeded into triplicate flasks and grown on plastic in adherence for 5-8 passages. Generation number accumulation was compared to a control line (transfection with Cas9 only, no sgRNA; Ctrl). Generation time number for porcine ADSC single edits is shown in graph (e), generation number for porcine ADSC double edits is shown in graph (f), and generation number for porcine ADSC triple edits is shown in graph (g).

FIG. 14. Editing efficiencies and growth data from bovine var. (variety) Angus myoblast cell lines. Cells were edited using one of three sgRNAs against bovine P53 (a), RB1 (b) or HRAS (c) respectively or using bP53 sgRNA1 and bRB1 sgRNA1 in combination (d) or as a triple edit using bP53 sgRNA3/bRB1 sgRNA3/HRAS sgRNA3 (e). Editing efficiencies (knockout for P53 and RB1; knockin for HRAS) were measured on three time points post editing. For a growth assay, cells were seeded into triplicate flasks and grown on Matrigel (f) or plastic (g) in adherence for 6 passages where possible. Generation number accumulation was compared to a control line (transfection with Cas9 only, no sgRNA; Ctrl).

FIG. 15. Editing efficiencies from bovine var. Angus adipose derived stem cells (ADSC) cell line. Cells were edited using bP53 sgRNA3 and bRB1 sgRNA2 in combination. Editing efficiencies (knockout) were measured on two time points post editing.

FIG. 16. Editing efficiencies and growth data from bovine var. Wagyu myoblast cell lines. Cells were edited using one sgRNA against P53 (a) and RB1 (b) respectively or both in combination (c). Editing efficiencies (knockout) were measured on three time points post editing. For a growth assay, cells were seeded into triplicate flasks and grown on Matrigel coated flasks or on plastic (d) in adherence for 5 passages. Doubling times were compared to a control line (transfection with Cas9 only, no sgRNA; Ctrl).

FIG. 17. Editing efficiencies from bovine var. Wagyu adipose derived stem cells (ADSC) cell lines. Cells were edited using one sgRNA against P53 (a) and RB1 (b) respectively or both in combination (c). Editing efficiencies (knockout) were measured on three time points post editing.

FIG. 18. Editing efficiencies from chicken myoblast cell lines. Cells were edited using one sgRNA against P53 (a) or one out of three sgRNAs against RB1 (b). Editing efficiencies (knockout) were measured on three time points post editing.

Table 1. Cell lines and specific growth conditions.

Table 2. CRISPR guide RNA sequences.

Table 3. PCR primers.

Table 4. Total editing and proportion of gene knock-out (KO) across a range of nucleofection conditions. Editing was assessed from inference of CRISPR edits from Sanger sequencing traces using the ICE tool (Conant et al., 2022). The R2 coefficient shows the goodness of fit of the ICE model and hence its reliability across the dataset for prediction of editing outcomes.

Table 5. Genes targeted and sequence of guide RNAs used with the Strep. pyogenes Cas9 protein.

Table 6. Sequence of template of porcine and bovine HRAS, which had a G12V mutation created by addition of a ssODN homology template (IDT technologies).

Table 7. Sequences of nucleic acids. These pig sequences include target sequences according to the invention and illustrate genetic modifications.

Table 8. H-RAS, N-RAS, and K-RAS amino acid sequences (pig).

### DETAILED DESCRIPTION OF THE INVENTION

The aspects of the invention will now be further described. In the following passages, different aspects are described. Each aspect so defined may be combined with any other aspect or aspects unless clearly indicated to the contrary.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012).

Enzymatic reactions and purification techniques are performed according to manufacturer's specifications, as commonly accomplished in the art or as described herein. The nomenclatures used in connection with, and the laboratory procedures and techniques of, cell biology and cell culturing, analytical chemistry, synthetic organic chemistry, and medicinal and pharmaceutical chemistry described herein are those well-known and commonly used in the art. Standard techniques are used for any cell culturing, genetic targeting, chemical syntheses, chemical analyses, and delivery.

Somatic cells isolated from tissues/organs often used in food consumption (e.g. muscle, fat, fibroblasts) from animal species for human or animal consumption, for example but not limited to agriculturally relevant species (e.g. pigs, cows, chickens) have a limited lifespan when grown in vitro (outside the originating animal). Through the manipulation of key regulatory genetic pathways in these cells, we have been able to immortalise a range of agriculturally-relevant cell types (e.g. muscle, fat, fibroblasts), which allows us to use these cells in cellular agriculture (where cells must proliferate for supra-physiological periods of time or indefinitely). We have achieved this through modulating key molecular pathways that involve genome surveillance, cell cycle and cell death controls (e.g. TP53, H-Ras and/or pRB pathways). We have shown that this can be done by modulating endogenous genes. We have shown that alteration of endogenous gene regulation/expression (e.g. TP53, RB1 and/or H-Ras) is both necessary and sufficient for immortalisation of a range of agriculturally-relevant cell types for human or animal consumption.

### Modified Cells and Methods

In a first aspect, the invention relates to an isolated modified non-human animal cell, cell population or cell culture having a genetic modification in one or more genes associated with genome surveillance, cell cycle control and/or cell death control. In one preferred embodiment, the animal is of an animal species suitable for human or animal consumption, for example, but not limited to an to agriculturally relevant animal species.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), naturally occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene”, “allele” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences. Thus, according to the various aspects of the invention, genomic DNA, cDNA or coding DNA may be used. In one aspect, the nucleic acid is cDNA or coding DNA. The terms “peptide”, “polypeptide” and “protein” are used interchangeably herein and refer to amino acids in a polymeric form of any length, linked together by peptide bonds. The term “allele” designates any of one or more alternative forms of a gene at a particular locus. Heterozygous alleles are two different alleles at the same locus. Homozygous alleles are two identical alleles at a particular locus. A wild type (wt) allele is a naturally occurring allele without a modification at the target locus.

According to the invention, one or more endogenous gene associated with genome surveillance, cell cycle control and/or cell death control is targeted to introduce the genetic modification. Suitable target genes in these pathways are listed below and include RB1, TP53, and/or a RAS gene family member, e.g. HRAS, NRAS, or KRAS. PTEN may also be targeted. Non-limiting example nucleic acid sequences and modifications therein from pig are provided in the examples and table 5.

According to the various aspects of the invention, the modification can be in the promoter region or in the coding region of the one of more genes that is/are targeted. The cell is therefore genetically manipulated/engineered. Preferably, the mutation is not naturally occurring. The inventors have demonstrated that manipulation of an endogenous gene or genes in non-human animal cells of interest can achieve immortalisation of cells without the expression of exogenous nucleic acids/proteins so that they can be used in cellular agriculture. In one embodiment, the modified cell does not express a foreign, i.e. exogenous nucleic acid construct. In particular, in another embodiment, the modified cell does not express a foreign, i.e. exogenous nucleic acid construct to manipulate expression or activity of components of the genome surveillance, cell cycle control and/or cell death control pathway. In particular, in another embodiment, the modified cell does not express an exogenous telomerase catalytic subunit (TERT). In particular, in another embodiment, the modified cell does not express exogenous an simian virus 40 (SV40) early region gene, exogenous TERT, exogenous mutant CDK4, and/or exogenous cyclin D1. Similarly, the methods of the invention do not include transfecting the cells with a plasmid or construct expressing an exogenous simian virus 40 (SV40) early region gene, exogenous TERT, exogenous mutant CDK4, and/or exogenous cyclin D1.

In one embodiment of the aspects of the invention, the modified cell is a primary cell. In another embodiment of the aspects of the invention, the modified cell is a somatic cell. Any somatic cell suitable for use in cellular agriculture, that is the production of animal-sourced foods from cell culture, is within the scope of the invention. For example, the cell may be a fat or muscle cell. For example, the cell may be selected from one or more of the following cell types: myoblast, fibroblast, myofibroblast, adipose derived stem cell, epithelial cell, mesenchymal stem cell, satellite cell or hepatocytes.

The terms “animal” and “non-human animal” with reference to animals and cells derived therefrom are used herein interchangeably and refer only to cells of non-human-animals. Cells for use in the invention may be of any other animal origin. However, the cells are not human cells. Cells suitable for use in cellular agriculture are preferably non-human animal cells that provide a source of any dietary protein, fat and/or carbohydrate.

The cells are cells of non-human animals that are suitable for human or animal consumption. These include animals such as non-human mammals, birds, fish, crustaceans, molluscs, reptiles, amphibians, or insects. Exemplary non-human mammals include those in the genera Bovinae, Camelidae, Canidae, Caprae, Cervidae, Felidae, Equidae, Lagomorphs, Macropodidae, Oves, Rodents, or Suidae. The cells may be cells of any livestock or poultry. The cells may be porcine, bovine (e.g. cattle), ovine, caprine, avine, or piscine. The cell may be shrimp, prawn, crab, crayfish, and/or lobster. In one embodiment, the animal is a pig or bovine (e.g. cattle).

The animal used in various aspects of the invention may be of an animal species used in agriculture. An animal species used in is an animal farmed for human. Such animals are listed above. In a preferred embodiment, they include pig, bovine (e.g. cattle), poultry (e.g. chicken, turkey, duck, geese), sheep, goat, fish, crustaceans or mollusc.

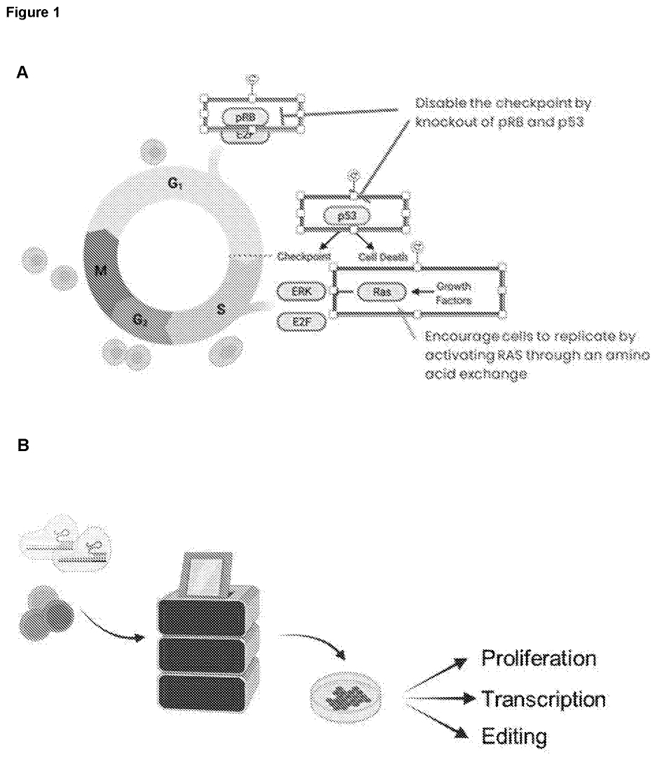

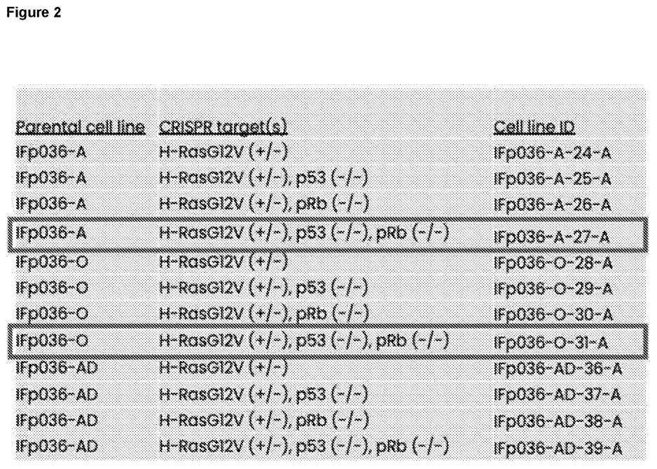

The genetic modification is in one or more genes associated with genome surveillance, cell cycle control and/or cell death control. The cell cycle of a cell is the series of growth and development steps the cell undergoes between its formation by the division of a mother cell and division to make two new daughter cells. The cell cycle is formed of a number of different phases with each phase having a number of steps which must be completed before moving on to the next phase of the cell cycle. The phases of the cell cycle are G1, S, G2, and Mitotic phase (M) as shown in FIG. 1. To prevent uncontrolled cell division, cell cycle checkpoints exist between cell cycle phases which ensure that the relevant steps of the particular cell cycle phase have been completed. If certain steps have not been completed or the proteins that control cell cycle checkpoints receive signals to prevent cell cycle progression the cell will not enter the next phase of the cell cycle. In an embodiment the invention provides a method by which the proteins responsible for cell cycle checkpoints are modified so that the cell is able to continue into the next phase of the cell cycle and thereby reduces the doubling time of the modified cell.

The doubling time of a cell line is the average time it takes for a population of the cells to double in size as a result of cell cycle progression and subsequent division. Therefore, removing cell cycle checkpoint inhibition of the cell cycle decreases the time required for one cell to undergo mitosis and form two new daughter cells. When applied to a whole population of cells of the cell line, this modification reduces the doubling time of said cell line and means the cells are quick to expand and better suited for use in cellular agriculture.

Immortalized cell lines are cells that have been manipulated to proliferate indefinitely and can thus be cultured for long periods of time.

The modified cells of the various aspects of the invention are capable of proliferating for a longer time than a wild type cell and can thus be cultured for long periods of time (longer compared to wild type), for example at least 60 doublings. They have the ability to be propagated in culture for extensive numbers of doublings. In one embodiment, the modified cells are immortalised and capable of proliferating indefinitely.

In one embodiment, the one or more genes is selected from one or more of RB1, TP53, and/or a RAS gene family member, e.g. HRAS. In a further related embodiment, the one or more genes is RB1. In another embodiment, the one or more genes is TP53. In another embodiment the one or more genes is a RAS gene e.g. HRAS. In another embodiment, the one or more genes is RB1 and TP53. In another embodiment, the one or more genes is RB1 and a RAS gene e.g. HRAS. In another embodiment, the one or more genes is TP53 and RAS e.g. HRAS. In another embodiment, the one or more genes is RB1 and TP53 and a RAS gene e.g. HRAS.

The term TP53 refers to the gene, TP53 to the protein.

In one embodiment of the various aspects of the invention, the RAS gene is HRAS, NRAS, or KRAS.

The term “genetic modification” relates to a modification that alters expression of the gene that is targeted or functional activity of the gene product, i.e. a gene associated with genome surveillance, cell cycle control and/or cell death control. The genetic modification may result in a loss of function, for example by creating a knock out. In another embodiment, the modification may be hyperactivation modification that increases activity of the expressed protein. To create a loss of function/knockout, a mutation may be introduced in the coding sequence which renders the expressed protein non-functional (e.g. an amino acid substitution, deletion or addition/insertion) or creates a premature stop codon/prevents expression of a functional protein. To create hyperactivation modification, a mutation may be introduced in the coding sequence which results in an amino acid substitution, deletion or addition in the protein sequence that renders the protein hyperactive.

Alternatively, a promoter sequence of the gene may be targeted to downregulate or upregulate expression.

In one embodiment, the modification in RB1 is a loss of function mutation. In one embodiment, the modification in TP53 is a loss of function mutation.

In another embodiment of the invention the gene is selected from one or both of RB1 and/or TP53 and the modification is a loss of function mutation. In a related embodiment, the loss of function modification comprises a knock-out of the gene.

Examples of loss of function mutations are described herein. However, any mutation that results in a dominant loss of function as described herein is encompassed within the scope of the invention. As used herein, “dominant” also encompasses “semi-dominant” or “partially dominant”. Therefore, the mutant allele may be fully dominant, partially dominant or semi-dominant. Preferably, the mutant allele is fully dominant. A loss of function mutation includes a knock-out modification or any other modification that causes an amino acid substitution or change wherein the substitution or change causes the resulting protein to lack a specific function or causes a reduction in the activity of said protein or prevents expression of the protein.

A knock-out modification or mutation may eliminate at least partially the specific endogenous nucleic acid sequence from the genomic DNA of the cell that codes for the protein of interest. By eliminating the corresponding nucleic acid sequence the protein can no longer be synthesised by the cellular machinery.

In a further embodiment of the invention, the gene is RAS and the modification is a hyperactivation modification. The RAS gene may be selected from any one of HRAS, NRAS, or KRAS. In a related embodiment of the invention, the hyperactivation modification comprises one or more amino acid substitutions in the protein. In a yet further related embodiment, the one or more amino acid substitution comprises substituting the glycine at position 12 of SEQ ID NO: 46, 47, or 48. In one embodiment, the one or more amino acid substitutions comprises substituting the glycine at position 12 of SEQ ID NO: 46, 47, or 48, wherein the one or more amino acid is selected from a list comprising alanine, cysteine, aspartic acid, arginine, serine, and valine. In one embodiment, the one or more amino acid substitutions comprises substituting the glycine at position 12 of SEQ ID NO: 46, 47, or 48, wherein the one or more amino acid is selected from a list consisting of alanine, cysteine, aspartic acid, arginine, serine, and valine. In one embodiment, the one or more amino acid substitutions comprises substituting the glycine at position 12 of SEQ ID NO: 46, 47, or 48 with valine. In one embodiment, the one or more amino acid substitutions comprises substituting the glycine at position 13 of SEQ ID NO: 46, 47, or 48. In one embodiment, the one or more amino acid substitutions comprises substituting the glycine at position 13 of SEQ ID NO: 46, 47, or 48, wherein the one or more amino acid is selected from a list comprising alanine, cysteine, aspartic acid, arginine, serine, and valine. In one embodiment, the one or more amino acid substitutions comprises substituting the glycine at position 13 of SEQ ID NO: 46, 47, or 48, wherein the one or more amino acid is selected from a list consisting of alanine, cysteine, aspartic acid, arginine, serine, and valine. In one embodiment, the one or more amino acid substitutions comprises substituting the glycine at position 13 of SEQ ID NO: 46, 47, or 48 with valine. In one embodiment, the one or more amino acid substitutions comprises substituting the glutamine at position 61 of SEQ ID NO: 46, 47, or 48. In one embodiment, the one or more amino acid substitutions comprises substituting the glycine at position 61 of SEQ ID NO: 46, 47, or 48, wherein the one or more amino acid is selected from a list comprising glutamic acid, histidine, lysine, proline, and arginine. In one embodiment, the one or more amino acid substitutions comprises substituting the glycine at position 61 of SEQ ID NO: 46, 47, or 48, wherein the one or more amino acid is selected from a list consisting of glutamic acid, histidine, lysine, proline, and arginine. In a yet further related embodiment, the one or more amino acid substitution comprises substituting the glycine at position 12 of SEQ ID NO: 1 with Valine.

A hyperactivation modification or mutation is a mutation or modification of the genomic DNA that codes for a specific protein of interest so that the resulting protein has an increased activity when synthesised by the cellular machinery. Increased activity means any activity that is higher than the normal activity of the protein when activated or inhibition of the protein is removed. Activity of a protein may be measured in any number of ways that are readily appreciated by the skilled person. One such measure is the turnover rate of the protein. Another method is measuring the rate of production of the reaction catalysed by the protein. A hyperactivation mutation does not necessarily increase the protein activity to a level higher than normal activity. A hyperactivation mutation may also remove any constitutive inhibition and/or inhibition of the protein as a result of binding to a second protein and/or protein complex so that the protein that is not normally constitutively active is constitutively active.

An amino acid substitution is effected by alterations in a nucleic acid sequence that results in the production of a different amino acid at a given site. This modification may affect the functional properties and/or activity of the encoded polypeptide or it may not affect the functional properties of the encoded polypeptide (conservative substitution). Conservative substitutions are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products. Non-conservative substitution leads to an encoded protein which does not retain the same functional properties and/or activity of the non-modified protein.

Suitable sequence from genes in pig (Sus scrofa domesticus) are described in table 5. Thus, the modified cell may be a pig cell and the targeted gene is selected from RB1, TP53, and/or HRAS. For example, exon 8 of RB1 (SEQ ID NO. 4) may be targeted. The modified exon may be as shown in SEQ ID No. 5 and/or 6. The wild type sequence of HRAS is shown in SEQ ID No. 1. A modified cell may comprise a modification in HRAS as shown in SEQ ID No. 2 and/or 3. The mutation in HRAS may comprise Gly>Val (aa12) [GGA>GTA] and optionally a PAM-blocking mutation Gly>Val (aa15) [GGG>GtG]. The wild type sequence of exon 5 of TP53 is shown in SEQ ID No. 10. A modified cell may comprise a modification in HRAS as shown in SEQ ID No. 11 and/or 12. Alternatives genes to HRAS are NRAS and KRAS.

The sequence in table 5 are from pig. However, the invention is not limited to modified pig cells. A skilled person would know that for the manipulation of other animal cells from animal species suitable for human or animal consumption, e.g. suitable for human consumption, e.g. suitable for animal consumption, e.g. used in agriculture, e.g. as listed herein, the equivalent orthologue, i.e. the endogenous RB1, TP53, and/or HRAS gene specific to the non-human animal species targeted is to be genetically modified. Suitable gene sequences can be identified from public databases. A skilled person would also be able to identify suitable sequences using standard methods in the art to identify homologs and orthologs, for example based on sequence identity with the pig sequences.

Sequence identity is commonly defined with reference to the algorithm GAP (Wisconsin GCG package, Accelerys Inc, San Diego USA). GAP uses the Needleman and Wunsch algorithm to align two complete sequences, maximising the number of matches and minimising the number of gaps. Generally, default parameters are used, with a gap creation penalty typically equaling 12 and a gap extension penalty equaling 4. Use of GAP may be preferred but other algorithms may be used, e.g. BLAST, or the Smith-Waterman algorithm, or the TBLASTN program, of, generally employing default parameters. In particular, the psi-Blast algorithm may be used. Sequence identity may be defined using the Bioedit, ClustalW algorithm. Alignments can be performed using Snapgene and based on MUSCLE (Multiple Sequence Comparison by Log-Expectation) algorithms.

In one embodiment, the modification is introduced using targeted genome modification and/or a rare-cutting endonuclease, for example a TALEN, ZFN or CRISPR/Cas9. However, other alternative endonucleases would be known to the skilled person.

Genome editing techniques have emerged as alternative methods to conventional mutagenesis methods (such as physical and chemical mutagenesis) or methods using the expression of transgenes in animal cells to produce mutant animal cells with improved phenotypes that are important in cellular research and cellular agriculture. These techniques employ sequence-specific nucleases (SSNs) including zinc finger nucleases (ZFNs), transcription activator-like effector nucleases (TALENs), and the RNA-guided nuclease Cas9 (CRISPR/Cas9), which generate targeted DNA double-strand breaks (DSBs), which are then repaired mainly by either error-prone non-homologous end joining (NHEJ) or high-fidelity homologous recombination (HR).

As explained in detail below, mutations according to the various aspects of the invention can be introduced into animal cells using targeted genome modification based on such editing techniques.

In another aspect, the invention also relates to a method for modifying the expression or function of one or more genes in a non-human animal wherein the gene is associated with genome surveillance, cell cycle control and/or cell death control. In a preferred embodiment, the animal is an animal suitable for human or animal consumption, for example used in agriculture. In an embodiment, the method comprises introducing a mutation into the one or more genes in the animal cell.

In another aspect, the invention relates to a method of producing a modified non-human animal cell described herein, wherein the method comprises introducing a genetic modification in one or more genes associated with genome surveillance, cell cycle control and/or cell death control. In a preferred embodiment, the animal is an animal used in agriculture. Suitable genes are described above. Embodiments defining various combinations, e.g. manipulation of all three genes, modifications made and cell types, are specifically set out elsewhere herein and apply to this aspect. The method is performed in vitro or ex vivo

In another aspect, the invention relates to a method of immortalising an animal cell, wherein the method comprises introducing a genetic modification in one or more genes associated with genome surveillance, cell cycle control and/or cell death control and wherein the animal is an animal suitable for human or animal consumption, for example used in agriculture. Suitable genes, animals and cells are described above. Embodiments defining various combinations, e.g. manipulation of all three genes, modifications made and cell types, are specifically set out elsewhere herein and apply to this aspect. The method may include the further step of culturing the cell in a suitable medium and propagating the cell. The method may include the further step of establishing a cell line. For example, a muscle cell may be grown in culture into muscle tissues that are attached to a support structure such as a two or three-dimensional scaffold or support structure. An immortalized animal cell obtained by the method is also within the scope of the invention. The method is performed in vitro or ex vivo.

In yet another aspect the invention provides a modified animal cell having a genetic modification in one or more genes selected from RB1, TP53 and/or a gene in the RAS family, e.g. H-RAS. Embodiments defining various combination, e.g. manipulation of all three genes, modifications made and cell types, are specifically set out elsewhere herein and apply to this aspect.

In all aspects of the invention the animal is not human. Animals that can be used, in particular agriculturally relevant animals, are listed herein.

### Targeted Genome Modification of Animal Cells Using Gene Editing

Targeted genome modification or targeted genome editing is a genome engineering technique that uses targeted DNA double-strand breaks (DSBs) to stimulate genome editing through homologous recombination (HR)-mediated recombination events. To achieve effective genome editing via introduction of site-specific DNA DSBs, four major classes of customizable DNA binding proteins can be used: meganucleases derived from microbial mobile genetic elements, ZF nucleases based on eukaryotic transcription factors, rare-cutting endonucleases/sequence specific endonucleases (SSN), for example TALENs, transcription activator-like effectors (TALEs) from Xanthomonas bacteria, and the RNA-guided DNA endonuclease Cas9 from the type II bacterial adaptive immune system CRISPR (clustered regularly interspaced short palindromic repeats). Meganuclease, ZF, and TALE proteins all recognize specific DNA sequences through protein-DNA interactions. Although meganucleases integrate their nuclease and DNA-binding domains, ZF and TALE proteins consist of individual modules targeting 3 or 1 nucleotides (nt) of DNA, respectively. ZFs and TALEs can be assembled in desired combinations and attached to the nuclease domain of FokI to direct nucleolytic activity toward specific genomic loci.

Upon delivery into host cells via the bacterial type III secretion system, TAL effectors enter the nucleus, bind to effector-specific sequences in host gene promoters and activate transcription. Their targeting specificity is determined by a central domain of tandem, 33-35 amino acid repeats. This is followed by a single truncated repeat of 20 amino acids. The majority of naturally occurring TAL effectors examined have between 12 and 27 full repeats.

These repeats only differ from each other by two adjacent amino acids, their repeat-variable di-residue (RVD). The RVD determines which single nucleotide the TAL effector will recognize: one RVD corresponds to one nucleotide, with the four most common RVDs each preferentially associating with one of the four bases. Naturally occurring recognition sites are uniformly preceded by a T that is required for TAL effector activity. TAL effectors can be fused to the catalytic domain of the FokI nuclease to create a TAL effector nuclease (TALEN) which makes targeted DNA double-strand breaks (DSBs) in vivo for genome editing. The use of this technology in genome editing is well described in the art, for example in U.S. Pat. Nos. 8,440,431, 8,440,432 and 8,450,471. Customized plasmids can be used with the Golden Gate cloning method to assemble multiple DNA fragments. The Golden Gate method uses Type IIS restriction endonucleases, which cleave outside their recognition sites to create unique 4 bp overhangs. Cloning is expedited by digesting and ligating in the same reaction mixture because correct assembly eliminates the enzyme recognition site. Assembly of a custom TALEN or TAL effector construct and involves two steps: (i) assembly of repeat modules into intermediary arrays of 1-10 repeats and (ii) joining of the intermediary arrays into a backbone to make the final construct.

Another genome editing method that can be used according to the various aspects of the invention is CRISPR. The use of this technology in genome editing is well described in the art, for example in U.S. Pat. No. 8,697,359. In short, CRISPR is a microbial nuclease system involved in defence against invading phages and plasmids. CRISPR loci in microbial hosts contain a combination of CRISPR-associated (Cas) genes as well as non-coding RNA elements capable of programming the specificity of the CRISPR-mediated nucleic acid cleavage. Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer).

By “crRNA” or CRISPR RNA is meant the sequence of RNA that contains the protospacer element and additional nucleotides that are complementary to the tracrRNA. By “tracrRNA” (transactivating RNA) is meant the sequence of RNA that hybridises to the crRNA and binds a CRISPR enzyme, such as Cas9 thereby activating the nuclease complex to introduce double-stranded breaks at specific sites within the genomic sequence of at least one nucleic acid or promoter sequence of the one or more genes. By “protospacer element” is meant the portion of crRNA (or sgRNA) that is complementary to the genomic DNA target sequence, usually around 20 nucleotides in length. This may also be known as a spacer or targeting sequence.

By “sgRNA” (single-guide RNA) is meant the combination of tracrRNA and crRNA in a single RNA molecule, preferably also including a linker loop (that links the tracrRNA and crRNA into a single molecule). “sgRNA” may also be referred to as “gRNA” and in the present context, the terms are interchangeable. The sgRNA or gRNA provide both targeting specificity and scaffolding/binding ability for a Cas nuclease. A gRNA may refer to a dual RNA molecule comprising a crRNA molecule and a tracrRNA molecule.

The Type II CRISPR is one of the most well characterized systems and carries out targeted DNA double-strand breaks in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA: tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Cas9 is thus the hallmark protein of the type II CRISPR-Cas system, and a large monomeric DNA nuclease guided to a DNA target sequence adjacent to the PAM sequence motif by a complex of two noncoding RNAs: CRIPSR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with a guide RNA (gRNA) also called single guide RNA (sgRNA) can introduce site-specific double strand breaks (DSBs) into genomic DNA of live cells from various organisms. For applications in eukaryotic organisms, codon optimized versions of Cas9, which is originally from the bacterium Streptococcus pyogenes, have been used.

Synthetic CRISPR systems typically consist of two components, the gRNA and a non-specific CRISPR-associated endonuclease and can be used to generate knock-out cells or animals by co-expressing a gRNA specific to the gene to be targeted and capable of association with the endonuclease Cas9. Notably, the gRNA is an artificial molecule comprising one domain interacting with the Cas or any other CRISPR effector protein or a variant or catalytically active fragment thereof and another domain interacting with the target nucleic acid of interest and thus representing a synthetic fusion of crRNA and tracrRNA. The genomic target can be any 20 nucleotide DNAsequence, provided that the target is present immediately upstream of a PAM sequence. The PAM sequence is of outstanding importance for target binding and the exact sequence is dependent upon the species of Cas9.

The PAM sequence for the Cas9 from Streptococcus pyogenes has been described to be “NGG” or “NAG” (Standard IUPAC nucleotide code) (Jinek et al, “A programmable dual-RNA-guided DNA endonuclease in adaptive bacterial immunity”, Science 2012, 337: 816-821). The PAM sequence for Cas9 from Staphylococcus aureus is “NNGRRT” or “NNGRR(N)”. Further variant CRISPR/Cas9 systems are known. Thus, a Neisseria meningitidis Cas9 cleaves at the PAM sequence NNNNGATT. A Streptococcus thermophilus Cas9 cleaves at the PAM sequence NNAGAAW. Recently, a further PAM motif NNNNRYAC has been described for a CRISPR system of Campylobacter (WO 2016/021973). For Cpf1 nucleases it has been described that the Cpf1-crRNA complex, without a tracrRNA, efficiently recognize and cleave target DNA proceeded by a short T-rich PAM in contrast to the commonly G-rich PAMs recognized by Cas9 systems. Furthermore, by using modified CRISPR polypeptides, specific single-stranded breaks can be obtained. The combined use of Cas nickases with various recombinant gRNAs can also induce highly specific DNA double-stranded breaks by means of double DNA nicking. By using two gRNAs, moreover, the specificity of the DNA binding and thus the DNA cleavage can be optimized. Further CRISPR effectors like CasX and CasY effectors originally described for bacteria, are meanwhile available and represent further effectors, which can be used for genome engineering purposes (Burstein et al., “New CRISPR-Cas systems from uncultivated microbes”, Nature, 2017, 542, 237-241).

Once expressed, the Cas9 protein and the gRNA form a ribonucleoprotein complex through interactions between the gRNA “scaffold” domain and surface-exposed positively-charged grooves on Cas9. Cas9 undergoes a conformational change upon gRNA binding that shifts the molecule from an inactive, non-DNA binding conformation, into an active DNA-binding conformation. Importantly, the “spacer” sequence of the gRNA remains free to interact with target DNA. The Cas9-gRNA complex will bind any genomic sequence with a PAM, but the extent to which the gRNA spacer matches the target DNA determines whether Cas9 will cut. Once the Cas9-gRNA complex binds a putative DNA target, a “seed” sequence at the 3′ end of the gRNA targeting sequence begins to anneal to the target DNA. If the seed and target DNA sequences match, the gRNA will continue to anneal to the target DNA in a 3′ to 5′ direction (relative to the polarity of the gRNA).

CRISPR/Cas9 and likewise CRISPR/Cpf1 and other CRISPR systems are highly specific when gRNAs are designed correctly, but especially specificity is still a major concern, particularly for clinical uses based on the CRISPR technology. The specificity of the CRISPR system is determined in large part by how specific the gRNA targeting sequence is for the genomic target compared to the rest of the genome. The sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp.

Thus, as used herein, the term “guide RNA” relates to a synthetic fusion of two RNA molecules, a crRNA (CRISPR RNA) comprising a variable targeting domain, and a tracrRNA. In one embodiment, the guide RNA comprises a variable targeting domain of 12 to 30 nucleotide sequences and a RNA fragment that can interact with a Cas endonuclease.

sgRNAs suitable for use in the methods of the invention are described below. As used herein, the term “guide polynucleotide”, relates to a polynucleotide sequence that can form a complex with a Cas endonuclease and enables the Cas endonuclease to recognize and optionally cleave a DNA target site. The guide polynucleotide can be a single molecule or a double molecule. The guide polynucleotide sequence can be an RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also contemplated.

The terms “target site”, “target sequence”, “target DNA”, “target locus”, “genomic target site”, “genomic target sequence”, and “genomic target locus” are used interchangeably herein and refer to a polynucleotide sequence in the genome (including choloroplastic and mitochondrial DNA) of a cell at which a double-strand break is induced in the cell genome by a Cas endonuclease. The target site can be an endogenous site in the genome, or alternatively, the target site can be heterologous to the plant and thereby not be naturally occurring in the genome, or the target site can be found in a heterologous genomic location compared to where it occurs in nature. As used herein, terms “endogenous target sequence” and “native target sequence” are used interchangeably herein to refer to a target sequence that is endogenous or native to the genome and is at the endogenous or native position of that target sequence in the genome.

The length of the target site can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs.

In one embodiment, the Cas endonuclease gene is a Cas9 endonuclease, such as but not limited to, Cas9 genes listed in WO2007/025097 incorporated herein by reference. In another embodiment, the Cas endonuclease gene is animal optimized Cas9 endonuclease.

In one embodiment, the Cas endonuclease gene is an animal codon optimized Streptococcus pyogenes Cas9 gene that can recognize any genomic sequence of the form N(12-30)NGG can in principle be targeted.

In one embodiment, the Cas endonuclease is introduced directly into a cell by any method known in the art, for example, but not limited to transient introduction methods, transfection and/or topical application.

Cas9 expression plasmids for use in the methods of the invention can be constructed as described in the art.

In one embodiment, targeted genome modification according to the various aspects of the invention comprises the use of a rare-cutting endonuclease, for example a TALEN, ZFN or CRISPR/Cas; e.g. CRISPR/Cas9. Rare-cutting endonucleases/sequence specific endonucleases are naturally or engineered proteins having endonuclease activity and are target specific. These bind to nucleic acid target sequences which have a recognition sequence typically 12-40 bp in length. In one embodiment, the SSN is selected from a TALEN. In another embodiment, the SSN is selected from CRISPR/Cas9. This is described in more detail below.

In one embodiment, the step of introducing a mutation comprises contacting a population of animal cells with DNA binding protein targeted to one or more endogenous RB1, and/or TP53, and/or RAS gene sequences, for example selected from the exemplary sequences listed herein. In one embodiment, the method comprises contacting a population of cells with one or more rare-cutting endonucleases; e.g. ZFN, TALEN, or CRISPR/Cas9, targeted to one or more endogenous RB1, and/or TP53, and/or a RAS gene sequences.

The method may further comprise the steps of selecting, from said population, a cell in which a RB1, and/or TP53, and/or a RAS gene sequence has been modified and regenerating said selected animal cell.

In an embodiment, the method comprises the use of CRISPR/Cas9. In this embodiment, the method therefore comprises introducing and co-expressing in an animal cell Cas9 and sgRNA targeted to one or more of RB1, and/or TP53, and/or a RAS gene sequences and screening for induced targeted mutations in one or more of RB1, and/or TP53, and/or RAS nucleic genes. The method may also comprise the further step of culturing the animal cells and selecting or choosing an animal cell with an altered cell cycle control phenotype, e.g. having reduced cell cycle checkpoint or increased cell cycle progression.

Cas9 and sgRNA may be comprised in a single or two expression vectors. The target sequence is one or more of RB1, and/or TP53, and/or a RAS nucleic acid sequence as shown herein.

In one embodiment, screening for CRISPR-induced targeted mutations in one or more RB1, and/or TP53, and/or RAS genes comprises obtaining a DNA sample from a transformed animal cell and carrying out DNA amplification and optionally restriction enzyme digestion to detect a mutation in one or more RB1, and/or TP53, and/or a RAS gene.

In one embodiment, the restriction enzyme is mismatch-sensitive T7 endonuclease. T7E1 is an enzyme that is specific to heteroduplex DNA caused by genome editing.

PCR fragments amplified from the transformed animal cells are then assessed using a gel electrophoresis assay based assay. In a further step, the presence of the mutation may be confirmed by sequencing the one or more RB1, and/or TP53, and/or RAS genes. Genomic DNA (i.e. wt and mutant) can be prepared from each sample, and DNA fragments encompassing each target site are amplified by PCR. The PCR products are digested by restriction enzymes as the target locus includes a restriction enzyme site. The restriction enzyme site is destroyed by CRISPR- or TALEN-induced mutations by NHEJ or HR, thus the mutant amplicons are resistant to restriction enzyme digestion, and result in uncleaved bands. Alternatively, the PCR products are digested by T7E1 (cleaved DNA produced by T7E1 enzyme that is specific to heteroduplex DNA caused by genome editing) and visualized by agarose gel electrophoresis. In a further step, they are sequenced.

In one embodiment, the method uses the sgRNA (and template, synthetic single-strand DNA oligonucleotides (ssDNA oligos) or donor DNA) constructs defined in detail below to introduce a targeted SNP or mutation, in particular one of the substitutions described herein into a GRF gene and/or promoter. The introduction of a template DNA strand, following a sgRNA-mediated snip in the double-stranded DNA, can be used to produce a specific targeted mutation (i.e. a SNP) in the gene using homology directed repair. Synthetic single-strand DNA oligonucleotides (ssDNA oligos) or DNA plasmid donor templates can be used for precise genomic modification with the homology-directed repair (HDR) pathway. Homologous recombination is the exchange of DNA sequence information through the use of sequence homology. Homology-directed repair (HDR) is a process of homologous recombination where a DNA template is used to provide the homology necessary for precise repair of a double-strand break (DSB). CRISPR guide RNAs program the Cas9 nuclease to cut genomic DNA at a specific location. Once the double-strand break (DSB) occurs, the mammalian cell utilizes endogenous mechanisms to repair the DSB. In the presence of a donor DNA, either a ssDNA oligo or a plasmid donor, the DSB can be repaired precisely using HDR resulting in a desired genomic alteration (insertion, removal, or replacement).

Single-strand DNA donor oligos are delivered into a cell to insert or change short sequences (SNPs, amino acid substitutions, epitope tags, etc.) of DNA in the endogenous genomic target region

A “donor sequence” is a nucleic acid sequence that contains all the necessary elements to introduce the specific substitution into a target sequence, preferably using homology-directed repair (HDR). In one embodiment, the donor sequence comprises a repair template sequence for introduction of at least one SNP. Preferably the repair template sequence is flanked by at least one, preferably a left and right arm, more preferably around 100 bp each that are identical to the target sequence. More preferably the arm or arms are further flanked by two gRNA target sequences that comprise PAM motifs so that the donor sequence can be released by Cas9/gRNAs. Donor DNA has been used to enhance homology directed genome editing (e.g. Richardson et al, Enhancing homology-directed genome editing by catalytically active and inactive CRISPR-Cas9 using asymmetric donor DNA, Nature Biotechnology, 2016 March; 34(3): 339-44).

The methods above use animal cell transformation to introduce an expression vector comprising a sequence-specific nucleases into an animal cell to target a GBP1 nucleic acid sequence. The term “introduction” or “transformation” as referred to herein encompasses the transfer of an exogenous polynucleotide into a host cell, irrespective of the method used for transfer.

Advantageously, any of several transformation methods may be used to introduce the gene of interest into a suitable cell. The methods described for the transformation of animal cells may be utilized for transient or for stable transformation. Transformation methods include the use of liposomes, electroporation, chemicals that increase free DNA uptake, injection of the DNA directly into the animal cell, particle bombardment as described in the examples, transformation using viruses or microinjection. Methods may be selected from the calcium/polyethylene glycol method for protoplasts, electroporation of protoplasts, microinjection into animal material, DNA or RNA-coated particle bombardment, infection with (non-integrative) viruses and the like.

Following DNA transfer and regeneration, putatively transformed animal cells may also be evaluated, for instance using Southern analysis, for the presence of the gene of interest, copy number and/or genomic organisation. Alternatively or additionally, expression levels of the newly introduced DNA may be monitored using Northern and/or Western analysis, both techniques being well known to persons having ordinary skill in the art.

The sequence-specific nucleases are preferably introduced into an animal cell as part of an expression vector. The vector may contain one or more replication systems which allow it to replicate in host cells. Self-replicating vectors include plasmids, cosmids and virus vectors. Alternatively, the vector may be an integrating vector which allows the integration into the host cell's chromosome of the DNA sequence. The vector desirably also has unique restriction sites for the insertion of DNA sequences. If a vector does not have unique restriction sites it may be modified to introduce or eliminate restriction sites to make it more suitable for further manipulation. Vectors suitable for use in expressing the nucleic acids, are known to the skilled person and a non-limiting example is pcDNA3.1. The nucleic acid is inserted into the vector such that it is operably linked to a suitable animal active promoter. Suitable animal active promoters for use with the nucleic acids include, but are not limited to PGK, CMV, EF1a, CAG, SV40 and Ubc.

In an embodiment of the invention the modification is made to the promoter region or coding region of the one or more genes.

### Cultivated Meat Products and Methods

As previously explained, the modified cells and methods for producing cells/methods for immortalizing an animal cells find use in cellular agriculture, i.e. the production of animal-sourced foods from cell culture.

Thus, in another aspect, the invention provides a method of producing cultivated meat/a cultivated meat product/food product comprising culturing a modified cell according to any previous embodiments of the invention. In a related embodiment, the method comprises carrying out continuous or batch culture of the modified cell.

The term “cultivated meat” is used herein to describe meat grown from in vitro animal cell culture distinguished from meat of slaughtered animals. Additional terms that may be used in the Art to describe meat grown from in vitro animal cell culture include cultured meat, cell-grown meat, clean meat, lab-grown meat, test tube meat, in vitro meat, tube steak, synthetic meat, cell-cultured meat, cell grown meat, tissue engineered meat, engineered meat, artificial meat, and manmade meat. The phrases “cell-based meat”, “slaughter-free cell-based meat”, “in vitro produced meat”, “in vitro cell-based meat”, “cultured meat”, “slaughter-free cultured meat”, “in vitro produced cultured meat”, “in vitro meat”, “in vitro cultured meat” and other similar such phrases are interchangeably used herein, and refer to the meat that is generated in vitro, starting with cells in culture, and that method which does not involve the slaughter of an animal in order to directly obtain meat from that animal for dietary consumption. The modified cells of the invention may be suitable for human and/or non-human consumption. In some embodiments, the cell-based meat is suitable for consumption by animals, such as domesticated animals. Accordingly, the cellular biomass herein support the growth of “pet food”, e.g. dog food, cat food, and the like.

Batch culture refers to culturing cells in a closed system whereby the culture of cells is carried out for a defined period of time or until a defined criteria is met. Once this criteria or time is met the culture is stopped, the cells harvested and the system emptied and cleaned ready for a new culture. The nutrients and/or culture additives may be added at the beginning of culture or during the culture. Continuous culture refers to culturing cells in a system whereby cells are continuously removed after a period of growth, or removed at specific points in time, while a population of cells remain in the system which are able to continue to grow and divide. This process is repeated for a set period of time or indefinitely. The nutrients and/or culture additives are added periodically or continuously so that the cells present in the system always have optimum conditions in which to grow and divide.

In another embodiment the invention provides cultivated animal tissue comprising the modified cell according to any previous embodiments of the invention.

In another aspect, the invention provides the use of the modified animal cell according to any previous embodiments for cellular agriculture.

In another aspect, the invention provides a method for producing an immortalised cell line comprising a method according to any previous aspects of the invention. This cell line can be used in cellular agriculture.

In a further aspect, the invention provides a method of producing a cultured meat product comprising culturing the one or more modified animal non-human cells or cell line according to any previous embodiments and optionally forming the cells into a tissue like structure. In a related embodiment, the method comprises forming the cells into a muscle tissue like structure. In a further aspect, the invention provides a cultured meat product for human or non-human consumption comprising a modified cell or cell line of the invention. In one embodiment, the animal for consumption is selected from a pig, bovine, poultry, sheep, goat, Equidae, fish, crustaceans or mollusc.

In a particular embodiment, a cultured meat product refers to a product in which cells according to the invention are formed into a product that is acceptable and/or suitable and/or appropriate for human consumption. The product may be of a structure that mimics or is intended to mimic the tissue of animal species which are used for human consumption. The cultured meat product may have a tissue like structure. The tissue may be selected from one or more of the following: muscle, fat, heart, liver, kidney and/or any tissue that is used for human consumption.

A tissue like structure according to the invention is a structure that resembles the specific tissue of an animal in terms of texture, taste, mouthfeel, visual structure, visual texture and colour. The tissue like structure does not have to be able to carry out the bodily functions that the tissue would carry out in vivo.

Tissue like structure is intended to mean that the tissue like structure appears similar or the same as tissue taken from the animal to a consumer of the cultured meat product.

The cultured meat product comprises modified cells according to the invention but may additionally comprise other components such as colourant, flavourings and/or flavour enhancing compositions and dietary supplements such as vitamins and/or minerals.

Also provided is a packaged cultivated meat product comprising or derived from a cell or cell line of the invention.

### Guide RNA and Kits

In one aspect of the invention, there is provided a guide RNA for use in a method of producing the modified, immortalised cell, or immortalised animal cell line as described herein.

In another aspect, the invention provides a guide RNA comprising any guide RNA selected from SEQ ID NO. 15, 16, 17, 18. In a further embodiment the invention provides a guide RNA according to any previous embodiment of the invention for use in a method of producing a modified cell according to any previous embodiment of the invention. In a related embodiment the invention provides a guide RNA according to the previous embodiment of the invention wherein the modified cell is a modified cell according to any previous embodiment of the invention.

In a further embodiment the invention provides a kit of parts comprising the guide RNA according as described above.

As explained above, in some embodiments, the methods of the invention use gene editing using sequence specific endonucleases that target one or more genes in an animal cell of interest. As also explained, Cas9 and gRNA may be comprised in a single or two expression vectors. The sgRNA targets the one or more gene nucleic acid sequence.

Thus, in another aspect of the invention, there is provided a nucleic acid construct comprising a nucleic acid sequence encoding at least one DNA-binding domain that can bind to the one or more genes. The one or more genes comprises of any sequence selected from SEQ ID NOs. 1, 4, 7, 10 or a functional variant, homolog or orthologue thereof as explained herein.

In one embodiment, the nucleic acid sequence encodes at least one protospacer element.

In one embodiment, the construct further comprises a nucleic acid sequence encoding a CRISPR RNA (crRNA) sequence, wherein said crRNA sequence comprises the protospacer element sequence and additional nucleotides. In one embodiment, the construct further comprises a nucleic acid sequence encoding a transactivating RNA (tracrRNA).

In a further embodiment, the construct encodes at least one single-guide RNA (sgRNA), wherein said sgRNA comprises the tracrRNA sequence and the crRNA sequence, wherein the sgRNA comprises or consists of a sequence selected from any of SEQ IDs 15, 16, 17, 18 listed herein, depending on the species targeted. PAM sequences are also shown in the in the section entitled sequences listing. The sgRNA can be used for manipulation of animal cells. In another aspect of the invention, there is provided a nucleic acid construct comprising a DNA donor nucleic acid wherein said DNA donor nucleic acid is operably linked to a regulatory sequence. The regulatory sequence may be one or more of the following: intron, promoter and/or terminator.

Cas9 and sgRNA may be combined or in separate expression vectors (or nucleic acid constructs, such terms are used interchangeably). Similarly, Cas9, sgRNA and the donor DNA sequence may be combined or in separate expression vectors. In other words, in one embodiment, an isolated animal cell is transfected with a single nucleic acid construct comprising both sgRNA and Cas9 or sgRNA, Cas9 and the donor DNA sequence as described in detail above. In an alternative embodiment, an isolated animal cell is transfected with two or three nucleic acid constructs, a first nucleic acid construct comprising at least one sgRNA as defined above, a second nucleic acid construct comprising Cas9 or a functional variant or homolog thereof and optionally a third nucleic acid construct comprising the donor DNA sequence as defined above. The second and/or third nucleic acid construct may be transfected before, after or concurrently with the first and/or second nucleic acid construct. The advantage of a separate, second construct comprising a Cas protein is that the nucleic acid construct encoding at least one sgRNA can be paired with any type of Cas protein, as described herein, and therefore is not limited to a single Cas function (as would be the case when both Cas and sgRNA are encoded on the same nucleic acid construct).

In one embodiment, a construct as described above is operably linked to a promoter, for example a constitutive promoter.

In another embodiment, the nucleic acid construct further comprises a nucleic acid sequence encoding a CRISPR enzyme. Preferably, the CRISPR enzyme is a Cas protein. More preferably, the Cas protein is Cas9 or a functional variant thereof.

In an alternative embodiment, the nucleic acid construct encodes a TAL effector. Preferably, the nucleic acid construct further comprises a sequence encoding an endonuclease or DNA-cleavage domain thereof. More preferably, the endonuclease is FokI.

In another aspect of the invention there is provided a single guide (sg) RNA molecule wherein said sgRNA comprises a crRNA sequence and a tracrRNA sequence. In one embodiment, the sgRNA molecule may comprise at least one chemical modification, for example that enhances its stability and/or binding affinity to the target sequence or the crRNA sequence to the tracrRNA sequence. For example, the crRNA may comprise a phosphorothioate backbone modification, such as 2′-fluoro (2′-F), 2′-O-methyl (2′-O-Me) and S-constrained ethyl (cET) substitutions.

In a further embodiment, the nucleic acid construct may further comprise at least one nucleic acid sequence encoding an endoribonuclease cleavage site. Preferably the endoribonuclease is Csy4 (also known as Cas6f). Where the nucleic acid construct comprises multiple sgRNA nucleic acid sequences the construct may comprise the same number of endoribonuclease cleavage sites. In another embodiment, the cleavage site is 5′ of the sgRNA nucleic acid sequence. Accordingly, each sgRNA nucleic acid sequence is flanked by an endoribonuclease cleavage site. The term ‘variant’ refers to a nucleotide sequence where the nucleotides are substantially identical to one of the above sequences. The variant may be achieved by modifications such as insertion, substitution or deletion of one or more nucleotides. In a preferred embodiment, the variant has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% identity to any one of the above described sequences. In one embodiment, sequence identity is at least 90%. In another embodiment, sequence identity is 100%. Sequence identity can be determined by any one known sequence alignment program in the art.

The invention also relates to a nucleic acid construct comprising a nucleic acid sequence operably linked to a suitable animal promoter. A suitable animal promoter may be a constitutive or strong promoter or may be a tissue-specific promoter. In one embodiment, suitable animal promoters are selected from, but not limited to, PGK, CMV, EF1a, CAG, SV40 and Ubc.

The nucleic acid construct of the present invention may also further comprise a nucleic acid sequence that encodes a CRISPR enzyme. In a specific embodiment Cas9 is codon-optimised Cas9. In another embodiment, the CRISPR enzyme is a protein from the family of Class 2 candidate proteins, such as C2c1, C2C2 and/or C2c3. In one embodiment, the Cas protein is from Streptococcus pyogenes. In an alternative embodiment, the Cas protein may be from any one of Staphylococcus aureus, Neisseria meningitides or Streptococcus thermophiles.

The term “functional variant” as used herein with reference to Cas9 refers to a variant Cas9 gene sequence or part of the gene sequence which retains the biological function of the full non-variant sequence, for example, acts as a DNA endonuclease, or recognition or/and binding to DNA. A functional variant also comprises a variant of the gene of interest which has sequence alterations that do not affect function, for example non-conserved residues. Also encompassed is a variant that is substantially identical, i.e. has only some sequence variations, for example in non-conserved residues, compared to the wild type sequences as shown herein and is biologically active.

In a further embodiment, the Cas9 protein has been modified to improve activity. For example, in one embodiment, the Cas9 protein may comprise the D10A amino acid substitution, this nickase cleaves only the DNA strand that is complementary to and recognized by the gRNA. In an alternative embodiment, the Cas9 protein may alternatively or additionally comprise the H840A amino acid substitution, this nickase cleaves only the DNA strand that does not interact with the sRNA. In this embodiment, Cas9 may be used with a pair (i.e. two) sgRNA molecules (or a construct expressing such a pair) and as a result can cleave the target region on the opposite DNA strand, with the possibility of improving specificity by 100-1500 fold. In a further embodiment, the Cas9 protein may comprise a D1135E substitution. The Cas 9 protein may also be the VQR variant. Alternatively, the Cas protein may comprise a mutation in both nuclease domains, HNH and RuvC-like and therefore is catalytically inactive. Rather than cleaving the target strand, this catalytically inactive Cas protein can be used to prevent the transcription elongation process, leading to a loss of function of incompletely translated proteins when co-expressed with a sgRNA molecule. An example of a catalytically inactive protein is dead Cas9 (dCas9) caused by a point mutation in RuvC and/or the HNH nuclease domains.

In a further embodiment, a Cas protein, such as Cas9 may be further fused with a repression effector, such as a histone-modifying/DNA methylation enzyme or a Cytidine deaminase to effect site-directed mutagenesis. In the latter, the cytidine deaminase enzyme does not induce dsDNA breaks, but mediates the conversion of cytidine to uridine, thereby effecting a C to T (or G to A) substitution. These approaches may be particularly valuable to target glutamine and proline residues in gliadins, to break the toxic epitopes while conserving gliadin functionality.

In a further embodiment, the nucleic acid construct comprises an endoribonuclease. Preferably the endoribonuclease is Csy4 (also known as Cas6f) and more preferably a codon optimised csy4. In one embodiment, where the nucleic acid construct comprises a Cas protein, the nucleic acid construct may comprise sequences for the expression of an endoribonuclease, such as Csy4 expressed as a 5′ terminal P2A fusion (used as a self-cleaving peptide) to a Cas protein, such as Cas9.

In one embodiment, the Cas protein, the endoribonuclease and/or the endoribonuclease-Cas fusion sequence may be operably linked to a suitable animal promoter. Suitable animal promoters are already described above, but in one embodiment, may be PGK, CMV, EF1a, CAG, SV40 and Ubc.

Suitable methods for producing the CRISPR nucleic acids and vectors system are known, and for example are published in Ran et al 2013, Nat Protoc 8, 2281-2308 (2013).

In a further aspect of the invention, there is provided an isolated animal cell transfected with at least one nucleic acid construct as described herein. In one embodiment, the isolated animal cell is transfected with at least one nucleic acid construct as described herein and a second nucleic acid construct, wherein said second nucleic acid construct comprises a nucleic acid sequence encoding a Cas protein, preferably a Cas9 protein or a functional variant thereof. Preferably, the second nucleic acid construct is transfected before, after or concurrently with the first nucleic acid construct described herein.

In an alternative aspect of the invention, the nucleic acid construct comprises at least one nucleic acid sequence that encodes a TAL effector.

Preferably, the nucleic acid encoding the sgRNA and/or the nucleic acid encoding a Cas protein is integrated in a stable form.

Also included in the scope of the invention, is the use of the nucleic acid constructs (CRISPR constructs) described above or the sgRNA molecules in any of the above described methods. For example, there is provided the use of the above CRISPR constructs or sgRNA molecules to modulate the activity of one or more gene as described herein. In particular, as described herein, the CRISPR constructs may be used to create loss of function or hyperactivation alleles.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present disclosure, including methods, as well as the best mode thereof, of making and using this disclosure, the following examples are provided to further enable those skilled in the art to practice this disclosure. However, those skilled in the art will appreciate that the specifics of these examples should not be read as limiting on the invention, the scope of which should be apprehended from the claims and equivalents thereof appended to this disclosure. Various further aspects and embodiments of the present disclosure will be apparent to those skilled in the art in view of the present disclosure.

All documents mentioned in this specification are incorporated herein by reference in their entirety, including references to gene accession numbers, scientific publications and references to patent publications.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

### EXAMPLES

### Example 1: Editing of Isolated Primary Cells

### Method

### Porcine Cell Line Establishment

Skeletal muscle biopsies were resected from Sus scrofa domesticus (Landrace) dams using standard veterinary procedures and in accordance with ethical body guidelines.

Biopsies were dissociated in medium and filtered to yield a single cell solution. Cells were then cultured on appropriate surfaces, matrices and in defined media conditions depending on the phenotype of cells. Plate coating for each line are shown in Table 1. All cell lines were maintained at 37° C. with 5% CO₂.

TABLE 1
Cell lines and specific growth conditions.

| Cell line | Cell type | Plate coating |
|---|---|---|
| IFp036-A | Myoblast | Matrigel |
| IFp036-O | Myofibroblast | No coating |
| IFp036-AD | Myofibroblast | Vitronectin |
| IFp044-A | Adipose-derived stem cell | No coating |
| IFp044-CA | Adipose-derived stem cell | Vitronectin |
| IFp044-CB | Epithelial | Vitronectin |

Note that the terms IVY or IF at the start of the cell line names throughout the document are interchangeable.

### Genome Editing of Primary Cells

CRISPR single guide RNAs (Phosphorothionate-modified sgRNA, Table 2) were designed against H-RAS, RB1 and TP53 genes. A single stranded oligo nucleotide donor was designed alongside the H-RAS guide RNA to create a heterozygous knock-in (KI) mutation of H-RasG12V (5′-ATGGTCTGGCCACGCTGAATGCTGCTTCCTCCACTGCAGTCCTGTGCTGTGGCCTCCTGAGGAG CGATGACGGAGTATAAGCTGGTGGTGGTGGGCGCTGTAGGTGTGGTGAAGAGTGCCCTGACCAT CCAGCTTATCCAGAACCACTTTGTGGATGAGTACGACCCCACCATAGAGGTGAGCCTCCAGCCC GCATCCCG-3′ SEQ ID NO. 19).

TABLE 2
CRISPR guide RNA sequences.

| Guide | | | Target sequence | SEQ ID | |
|---|---|---|---|---|---|
| ID | Gene | Exon | (5′ > 3′ | NO. | PAM |
| psg284 | RB1 | 8 | CTCCTGTTCTGA | SEQ ID | TGG |
| | | | CCTCGCCT | NO. 15 | |
| psg286 | TP53 | 5 | CGCGGACACGGG | SEQ ID | GGG |
| | | | TGCCAGGC | NO. 16 | |
| psg289 | H-RAS | 2 | TGGTGGGCGCTG | SEQ ID | GGG |
| | | | GAGGTGTG | NO. 17 | |
| psg173 | PTEN | 2 | AAAGACTTGAAG | SEQ ID | AGG |
| | | | GCGTATAC | NO. 18 | |

The isolated primary cell lines (IIVY-p036-A, O, AD and IFp044-A and C) were electroporated (Amax 4D Nucleofector, Lonza) with ribonucleoprotein (RNP) complexes composed of TrueCut™ SpCas9 protein V2 (Invitrogen A36498) and guide RNA (sgRNA, Synthego) as per manufacturer's instructions (Lonza). IF-p036-A is a myoblast cell line, IF-p036-O is a fibroblast or myofibroblast cell line, IF-p036-AD is a fibroblast or myofibroblast cell line. IFp044-A and IFp044-C are adipose-derived stem cell lines (Table 1). Electroporated cells were reseeded and expanded for 3 days in 24 well tissue culture plates. Cells were harvested (day 3 post electroporation), a portion of the cells were re-seeded to grow for a further 7 and 13 days (day 10 and 16 post electroporation) and the remaining cells were pelleted for DNA extraction. A cell pellet was taken at 3, 10 and 17 days post-electroporation and genomic DNA was extracted (Qiagen, DNeasy blood and tissue kit, 69506). The extracted DNA (isolated at day 3, 10 and 17) were used as a template to amplify the regions around and including the target guide RNA/Cas9 editing site using PCR [internal region with H-Ras, TP53 and RB1]. The relevant Exon of each gene was amplified by PCR (Q5 High fidelity DNA polymerase, NEB cat #M0491S, Table 3). Amplicons were subjected to Sanger sequencing and analysed using the Synthego ICE web tool to calculate the percent editing. The DNA amplification products were gel purified and submitted for sanger sequencing. ICE analysis (Inference of CRISPR Edits) was used to analyse the sequencing data and define the efficiency of gene editing achieved for H-Ras, TP53 and RB1. The DNA was also checked for off targets in relation to H-Ras by PCR. The top 3 predicted putative off-target sites for sg289 were amplified and sequenced. No off-target editing was observed. Control lines were generated by electroporation with SpCas9 only (no sgRNA). All cell lines were confirmed negative for Mycoplasma by (Mycoplasma test) assay.

TABLE 3
PCR primers

| | Forward | | Reverse | | |
|---|---|---|---|---|---|
| | Primer | SEQ ID | Primer | SEQ ID | Size |
| Gene | (5′ > 3′) | NO. | (5′ > 3′) | NO. | bp |
| RB1 | AGTTGGGGGC | SEQ ID | GATTCCAGAG | SEQ ID | 577 |
| | AGAAGAGAAT | NO. 20 | TGAGGGTGCT | NO. 21 | |
| TP53 | CTTGGCCTCT | SEQ ID | CCACGCTGTG | SEQ ID | 627 |
| | CATCCTTCCC | NO. 22 | TCGAAAAGTG | NO. 23 | |
| H-Ras | CTTCTAATTC | SEQ ID | AATGACCACT | SEQ ID | 568 |
| | GGGTGCGTGC | NO. 24 | TGCTTCCGGT | NO. 25 | |
| PTEN | GGGGGTCGTT | SEQ ID | AAGCTAACAG | SEQ ID | 566 |
| | GTCTTTACCT | NO. 26 | CCAACTGTGA | NO. 27 | |
| | | | AA | | |

### Lentivirus Generation and Transduction

Lentiviral vectors were prepared as previously described (Dull et al 1998). Briefly, HEK293T cells were plated at a density of 4×10⁶ cells per 10 cm dish in DMEM-F12/10% FBS (Gibco). At 24 h post-seeding, the cells were transfected using Lipofectamine 2000 (Thermo Fisher Scientific) and packaging mix (10.7 μg of Lentiviral transfer plasmid, 4.2 μg pMDL, 2.1 μg of pCMV-Rev and 3 μg of pVSV-G) according to the manufacturer's instructions. Medium was exchanged 24 h post-transfection, and at 48 h and 72 h post-transfection virus containing supernatant was harvested. Harvested supernatant was pre-cleared by centrifugation at 470 g for 5 mins and passed through a 0.45 μm filter, then stored at −80° C. until use.

Cells were infected by overlaying between a 1:10 and a 1:4 dilution of viral supernatant (mixed with appropriate medium) for 48 h, followed by medium exchange. Successful infection was measured by fluorescent microscopy, flow cytometry or resistance to a relevant antibiotic.

### Microscopy

Images were captured either on the Nikon Eclipse TS100 with CoolLED pE-300lite.

### Cell Counting

Cells were counted either using a haemocytometer with Trypan-Blue exclusion dye or counted using the cell counting machines K2, Celigo or NC3000. Doubling times were calculated using the doubling time web tool (https://www.doubling-time.com/compute.php).

### Results

### Simultaneous Multiplex Gene Silencing is Highly Efficient in Myoblast-Derived Porcine Cells

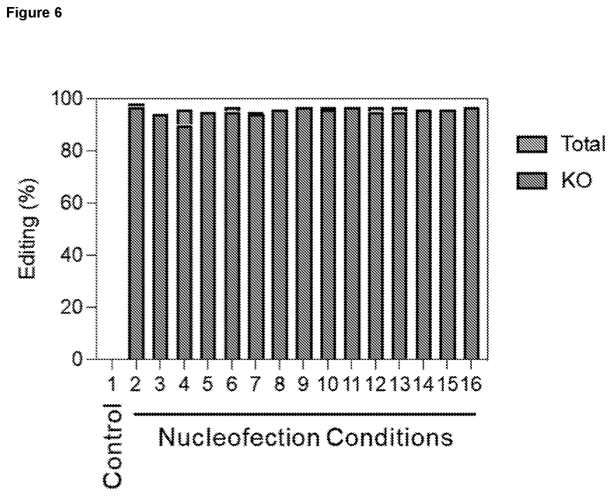

To understand the extent to which porcine cells can be immortalised without the addition of exogenous genes, and without over-expression of TERT, we first sought to optimise the parameters necessary for gene silencing using CRISPR-Cas9 editing. Immortal porcine myoblast-derived cells (vi-pMyoHet) were nucleofected with a Control sgRNA targeting the PTEN gene across a range of nucleofection conditions and the presence of successful editing was measured using amplicon sequencing (FIG. 6 and Table 4).

TABLE 4
Total editing and proportion of gene knock-out (KO) across a
range of nucleofection conditions. Editing was assessed from
inference of CRISPR edits from Sanger sequencing traces using
the ICE tool(Conant et al., 2022). The R2 coefficient shows
the goodness of fit of the ICE model and hence its reliability
across the dataset for prediction of editing outcomes.

| | | Total Editing | Knock-Out | R² |
|---|---|---|---|---|
| Condition | Pulse code | (%) | (%) | coefficient |
| 1 | Negative | NA | NA | NA |
| 2 | CA137 | 98 | 97 | 0.98 |
| 3 | CM138 | 94 | 94 | 0.94 |
| 4 | CM137 | 96 | 90 | 0.96 |
| 5 | CM150 | 95 | 95 | 0.95 |
| 6 | DN100 | 97 | 95 | 0.97 |
| 7 | DS138 | 95 | 94 | 0.95 |
| 8 | DS137 | 96 | 96 | 0.96 |
| 9 | DS130 | 97 | 97 | 0.97 |
| 10 | DS150 | 97 | 96 | 0.96 |
| 11 | DS120 | 97 | 97 | 0.97 |
| 12 | EH100 | 97 | 95 | 0.97 |
| 13 | EO100 | 97 | 95 | 0.97 |
| 14 | EN138 | 96 | 96 | 0.96 |
| 15 | EN150 | 96 | 96 | 0.96 |
| 16 | EW113 | 97 | 97 | 0.97 |

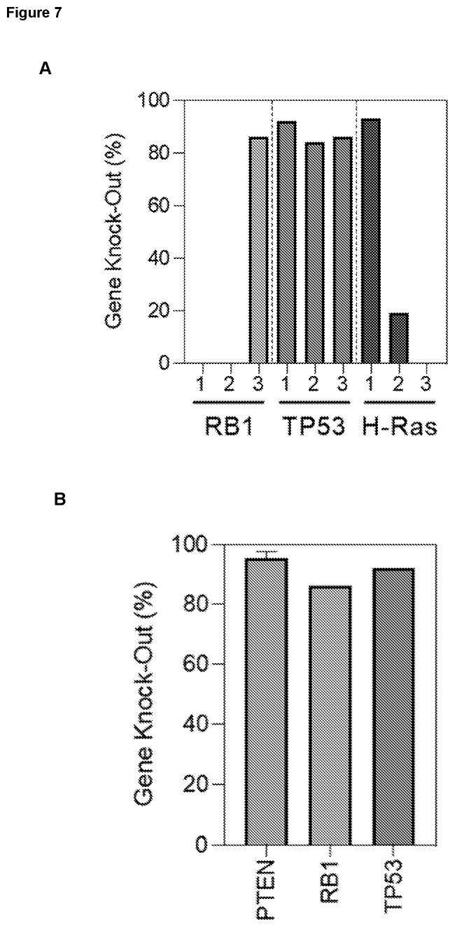

Three sgRNAs were designed against the RB1, TP53 and H-RAS genes and assessed for their ability to lead to gene KO in a second myoblast-derived line, vi-pMyoHom. Highly active sgRNAs were identified for each gene, with 86%, 92% and 93% KO respectively (FIG. 7, panel A). Multiplexing of these sgRNAs to simultaneously target all three genes yielded gene KO frequencies ranging from 86% to 96% KO (FIG. 7, panel B). Thus, these data demonstrate that it is possible to efficiently target three genes pivotal to cell cycle regulation concomitantly in myoblast-derived porcine cells.

Primary Porcine-Derived Myoblastic Cell Lines are Sensitive to Loss of H-Ras GTPase while TP53 and RB1 Loss Confer a Survival Advantage

The skilled person understands that the Ras family of small GTPases regulate cell division by converting cellular GTP to GDP and engaging the Raf/MEK/ERK pathway. There is redundancy between the three Ras subfamily members (H-Ras, N-Ras and K-Ras) with K-Ras−/−H-Ras−/− double mutant mice displaying a normal developmental phenotype due to compensation by K-Ras. Further, gain-of-function alterations, such as the substitution of Valine for Glycine at position 12 in SEQ ID NO. 46, render the protein into a constitutively active state, proving a strong mitogenic signal to drive cell division. Alternative substitutions of the Glycine at position 12 which produce the same advantageous gain-of-function are alanine, cysteine, aspartic acid, arginine, or serine. Substitutions at position 13 of SEQ ID NO: 46, 47, or 48 with any one of alanine, cysteine, aspartic acid, arginine, serine, or valine and/or substitutions at position 61 of SEQ ID NO: 46, 47, or 48 with any one of glutamic acid, histidine, lysine, proline, and arginine also produce the same advantageous gain-of-function.

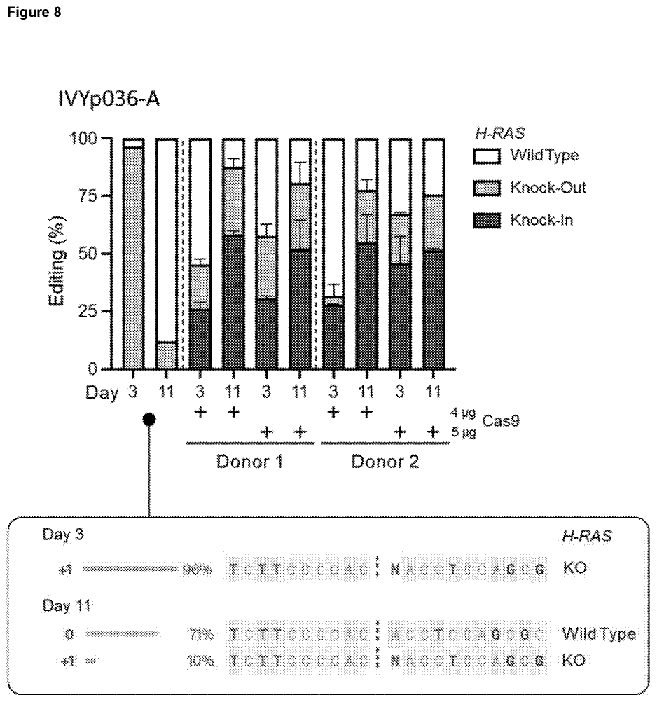

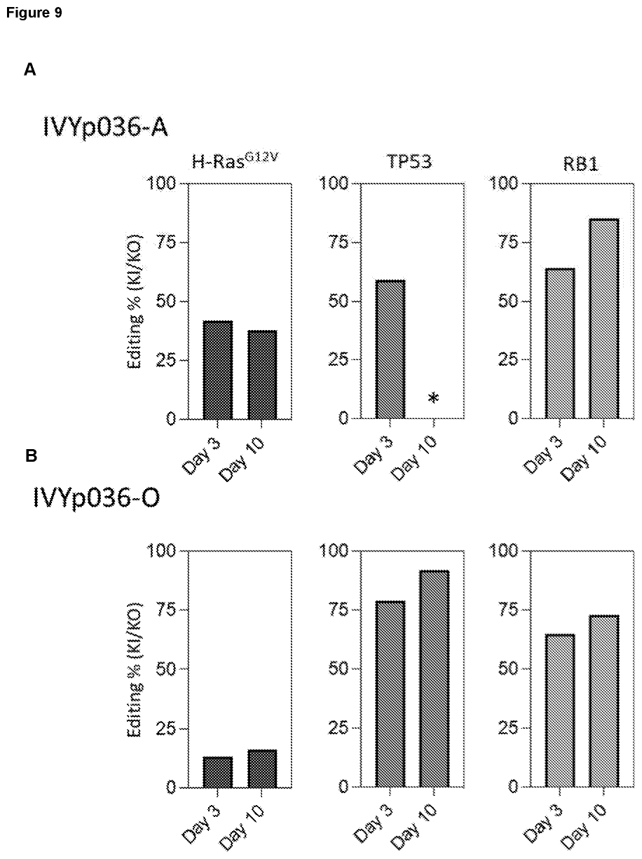

To explore the effects of H-Ras loss, and H-RasG12V gain-of-function, on primary porcine myoblast cells, RNP targeting the H-Ras gene was nucleofected, with and without a donor template containing the G12V variant. Acute biallelic disruption of the H-Ras gene led to a marked selective disadvantage of cells compared to unedited sister cells (FIG. 8). Upon 11 days of culture, H-Ras−/− were cells substantially out competed by H-Ras+/+ cells, resulting in a 10-fold depletion of H-Ras−/− cells. This negative selection of H-Ras loss suggests that compensation by other subfamily members is less pronounced in primary porcine myoblasts. Introduction of the H-RasG12V variant, however, demonstrated a different trend stabilised cells containing a loss of H-Ras cells. Presumably as the sequencing is performed upon a pool of cells, the surviving fraction of cells indeed encode a heterozygous H-RasG12V/− genotype (FIG. 8). Knock-in (KI) of the G12V substitution into two separate myoblast-derived porcine lines showed similar trends (FIG. 9). Inactivation of both TP53 and RB1 showed an enrichment after 10 days of culture, suggesting that loss of both proteins confers a survival advantage to primary porcine myoblasts (FIG. 9).

Generation of a Panel of Primary Myoblast- and Adipocyte-Derived Porcine Cell Lines with Differing Genetic Perturbations.

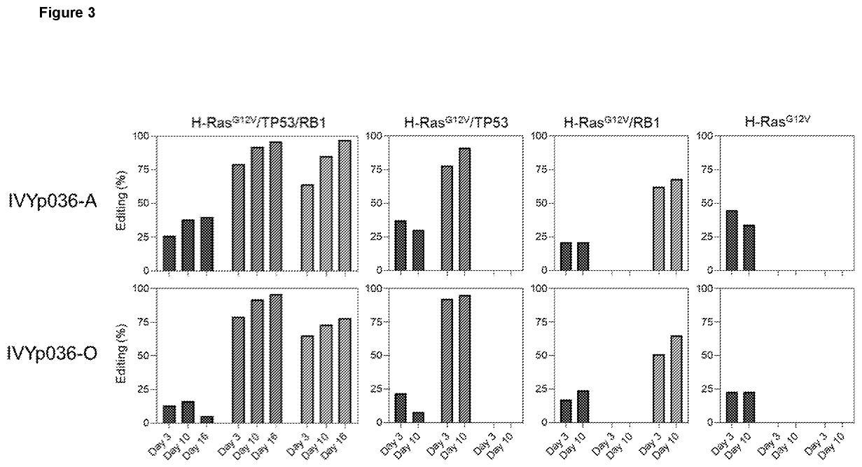

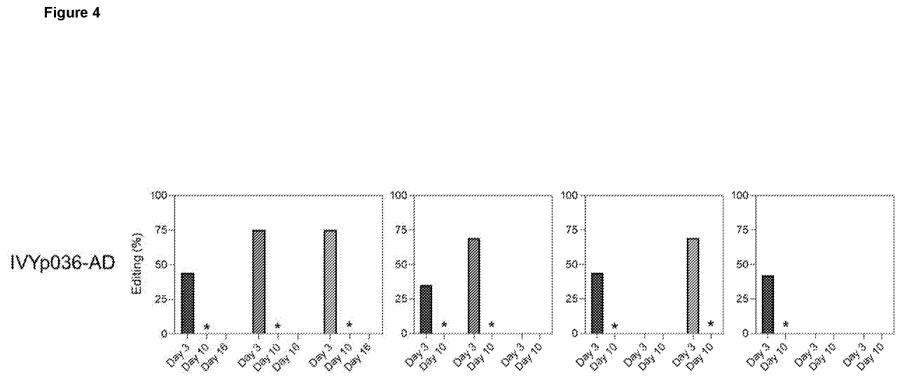

To understand the effects of perturbations on immortalisation across primary porcine cell types, two myoblast-derived (IVYp036-A and IVYp036-O) lines and one adipose-derived (IVYp036-AD) line were subjected to various combinations of CRISPR-Cas9 editing. All three lines were either single gene edited (H-RasG12V), dual gene edited (H-RasG12V/TP53 or H-RasG12V/RB1) or triple gene edited (H-RasG12V/TP53/RB1), then the edits were assessed overtime in culture (FIGS. 3 and 4).

FIGS. 3 and 4 show the relative efficiency of cellular editing of RAS, TP53 and RB1 in three primary porcine cell lines (IVYp036-A-27-A, IVYp036-O-31-A and IVYp036-AD). Each cell line was sampled at day three, ten and sixteen to determine the percentage of cells that possessed the desired modification. The percentage of successfully edited cells was calculated using Inference of CRISPR Edits (ICE). Both these edited cell lines demonstrate exceptionally high levels of editing.

As shown in FIGS. 3 and 4, all cell types demonstrated robust editing across all three genes. A trend was observed across both myoblast lines and all gene combinations whereby both TP53 and RB1 loss were selected for over time. Notably, there was no substantial enrichment for H-RasG12V over time across both myoblast lines.

FIG. 4 shows the relative efficiency of cellular editing of RAS, TP53 and RB1 in a third cell line (IFp036-AD) and demonstrates that this cell line also demonstrates exceptionally high levels of editing.

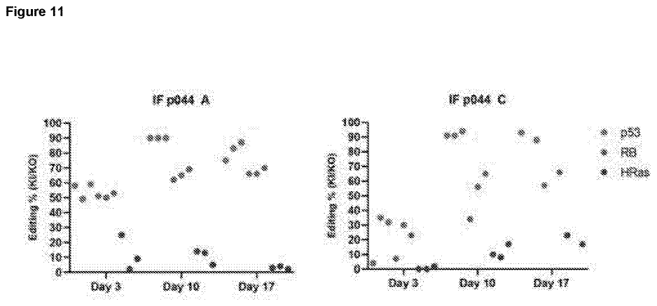

FIG. 11 shows the relative efficiency of RAS, TP53 and RB1 in two additional cells lines (IF p044 A and C) and demonstrates that these cell lines also demonstrate high levels of editing.

### Triple Edited (H-RasG12V/TP53/RB1) Primary Porcine Lines Display Molecular and Physical Hallmarks of Immortalisation

To explore whether triple editing (H-RasG12V/TP53/RB1) confers an immortal phenotype upon primary porcine cells, transcriptional profiling of cell cycle regulators and dependence on growth factors for proliferation were assessed.

Inactivation of TP53 signalling through genetic ablation of TP53 resulted in a marked reduction of p21 mRNA levels (FIG. 11). The p21 cyclin-dependent kinase (CDK) is a direct target of TP53 and a potent negative regulator of the cell cycle, at least in part by inhibiting CDK4,6/Cyclin-D, CDK2/Cyclin-E and Cyclin B. Activation of the p53-p21 signalling cascade can lead to cell cycle arrest or apoptosis. The Triple-edited CRISPR line demonstrated a robust de-repression of Cyclin B and a modest de-repression of CDK2 and CDK4, consistent with a relaxation of cell cycle regulatory controls. Importantly, the transcriptional changes of the triple-edited myoblast line mirrors closely that of the immortal myoblast control (vi-pMyoHet).

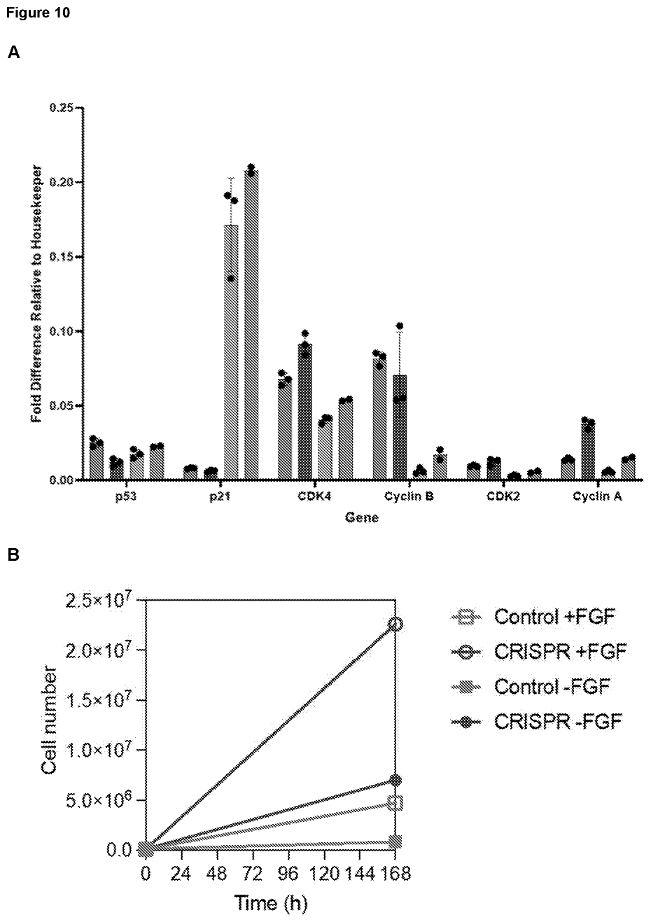

Consistent with a relaxation on cell cycle constraints, the triple-edited line demonstrated increased proliferation in comparison to the control, Cas9-nucleofected line (FIG. 10, panel B). Further, the triple-edited line was able to proliferate robustly in the absence of basic fibroblast growth factor (FGF2) supplementation whereas the non-immortalised line failed to proliferate. Taken together, these data indicate that immortalisation of primary porcine cells using CRISPR-Cas9 targeting of H-RasG12V, TP53 and RB1 pathways was successful.

### Discussion

To date, all approaches to immortalise cellular agriculture-relevant cell types have employed the addition of exogenous genes (eg CDK4) and over-expression of exogenously telomerase, TERT. Here the inventors have introduced no foreign genes into the cell line, nor utilising the over-expression of TERT. The inventors have shown that primary porcine muscle- and fat-derived cells can be efficiently gene edited using CRISPR-Cas9, and that editing of H-Ras, p53 and pRB pathways drives immortalisation.

Contrary to its neutral role in the mouse, inactivation of H-Ras had a detrimental effect on myoblast proliferation, allowing for the over-growth of wild type cells (FIG. 8). This suggests that the normal compensatory mechanisms (N-Ras and K-Ras) seen in the mouse do not act in the same way in primary porcine myoblast cells. Of note, inactivation of the Tp53 and RB1 pathways gave a competitive advantage to cells over controls (FIGS. 3 and 4). Without wishing to be bound by theory, this is as least in part due to the perturbation of cell cycle regulatory constraints enacted by these pathways (FIG. 10, panel A). Finally, it was noted that immortalisation through triple-gene editing both increased proliferation and relieved the reliance on exogenous growth factor (FGF2) addition to the culture medium (FIG. 10, panel B). Taken together, these data outline a robust strategy for the immortalisation of divergent cell types from agriculturally-relevant species and show that these edited cell lines will form the foundation for an economically viable large-scale production pipeline for the generation of porcine based meat products.

### Example 2: Doubling Times of Parental (IFp036-A/O), CRISPR Triple Edited and Cas9 Cell Lines

### Method

Cells were counted either using a haemocytometer with Trypan-Blue exclusion dye or counted using the K2, Celigo or NC3000. Doubling times were calculated using the doubling time web tool (https://www.doubling-time.com/compute.php).

### Results

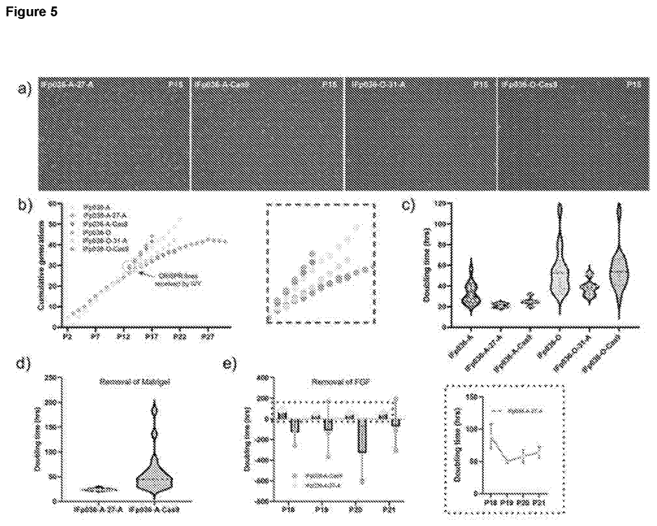

The characterisation of immortalisation was carried out using the triple edited cell lines IF-p036-A-27-A and IFp036-O-31-A compared to the non-edited parental control (denoted cas9 only). The triple edited cells show a growth advantage compared to Cas9 only (FIG. 5, panel c) as shown by the reduction in doubling time that the triple edited cell lines have compared to the Cas9 control cell line. A reduction in doubling time indicates that the modifications made to the cells have caused the cells to progress through the cell cycle quicker and as such display an immortalised phenotype.

The reduction in doubling time is more pronounced in the IFp036-O fibroblast/myofibroblast cell line compared to the IFp036-A myoblast cell line. However, in both cell lines there is a marked reduction in doubling time between the original cell line and the triple edited cell line and between the cas9 control cell line and the triple edited cell line.

### Example 3: Removal of Matrigel and the Effects on Doubling Time

### Method

The doubling time was assessed using the same method as above.

### Results

Matrigel is a commercially available matrix that contains extracellular matrix proteins and is used to aid the attachment and differentiation of cells in vitro. Attachment of cells to an extracellular matrix often works to keep primary derived cells in a quiescent state and promotes a longer doubling time. In addition, the reliance on cell attachment for cell survival is not advantageous for cultivated meat production where it is advantageous to cultivate non-attached cells which are easier to form into cultivated meat tissue. FIG. 5, panel d shows that the triple edited cells show a much reduced doubling time compared to the cas9 control cell line when the cells are culture in the absence of Matrigel. Therefore, by generating modified cells with an immortalised phenotype that do not rely on Matrigel the cells display a reduced doubling time and immortalised phenotype.

### Example 4: Removal of FGF and the Effects on Doubling Time

### Method

The doubling time was assessed using the method as detailed above.

### Results

FGF is s growth factor used to encourage cells cultured in vitro to grow and proliferate. The ability to culture cells in medium lacking this growth factor is advantageous to cells culture for cultivated meat as FGF is an expensive medium additive. A lower doubling time when cultured in medium lacking FGF is indicative of an immortalised phenotype as the cells do not require the stimulation to proliferate otherwise provided by FGF.

FIG. 5, panel e shows that the triple edited cell line demonstrates a reduced doubling time compared to the Cas9 control cell line highlighting that the triple edited cell line displays an immortalised phenotype compared to the Cas9 control cell line.

The final panel of FIG. 5, panel e demonstrates that the doubling time of the triple edited cell line reduces over a longer time in culture and with passages after passage 18. This is in direct contrast to the situation that primary cells usually display after a longer time being cultured in vitro. Non-edited primary cells would be expected to slow their growth and have an increased doubling time when cultured in vitro over a longer period. This difference highlights that the triple edited cell line has an immortalised phenotype compared to the Cas9 control and non-edited cell lines.

### Example 5: Editing Efficiencies and Growth Data from Porcine Myoblast Cell Lines

### Method

Cells were edited following the protocol described for the triple edited porcine Myoblast line. The genes in Table 5 were targeted using guide RNAs with the following sequences and using Strep. Pyogenes Cas9 protein. Note that the same guide RNAs were used between varieties of a given species (e.g. bovine var. Wagyu and var. Angus) as well as cell types (e.g. porcine myoblasts and porcine ADSC).

TABLE 5

| | Target | Target Gene | Guide ID | Protospacer sequence | Seq ID |
|---|---|---|---|---|---|
| Species | gene | Ensembl ID | (Species, ID) | (5′-3′) | NO. |
| Porcine | P53 | ENSSSCG00025036064 | psgRNA286 | CGCGGACACGGGTGCCAGGC | 28 |
| | RB1 | ENSSSCG00025015354 | psgRNA284 | CTCCTGTTCTGACCTCGCCT | 29 |
| | HRAS | ENSSSCG00025010749 | psgRNA289 | TGGTGGGCGCTGGAGGTGTG | 30 |
| Bovine | P53 | ENSBTAG00000001069 | bPS3 sgRNA1 | CGTCTAGGGTTCCTGCAATC | 31 |
| | | | bP53 sgRNA2 | ACTTGGCTGTTCCGGATTGC | 32 |
| | | | bP53 sgRNA3 | AGTCGACCCACAGCTGCACT | 33 |
| | RB1 | ENSBTAG00000006540 | bRB1 sgRNA1 | TATGGGTCTTTGAGCAATGC | 34 |
| | | | bRB1 sgRNAZ | CACCAAGGCGAGGTCAGAAC | 35 |
| | | | bRB1 sgRNA3 | TCTAGATGCAAGATTATTTC | 36 |
| | HRAS | ENSSTAG00000046644 | bHRAS sgRNA1 | GGCGCTCTTCCCCACGCCAC | 37 |
| | | | bHRAS sgRNA2 | CTCGTGGTGGTGGGCGCCGG | 38 |
| | | | bHRAS sgRNA3 | GGTGGTGGGCGCCGGTGGCG | 39 |
| Chicken | P53 | ENSGALG00010000434 | CP53 sgRNA3 | CTCACCATCCTTACACTGGA | 40 |
| | RB1 | ENSGALG00010003900 | CRB1 sgRNA1 | TATGGTTCCTTGAGCATAGC | 41 |
| | | | CRB1 sgRNA2 | CCCCACGAAGAGGTCAGAAC | 42 |
| | | | CRB1 sgRNA3 | TATAGATGCAAGATTATTTC | 43 |

For porcine and bovine HRAS, a G12V knockout was created by addition of a ssODN homology template (IDT technologies). The sequence of that template is given in Table 6.

TABLE 6

| | | Seq |
|---|---|---|
| | Homology template sequence | ID |
| Species | (5′-3′) | NO. |
| Porcine | ATGGTCTGGCCACGCTGAATGCTGCTTCCTC | 44 |
| | CACTGCAGTCCTGTGCTGTGGCCTCCTGAGG | |
| | AGCGATGACGGAGTATAAGCTGGTGGTGGTG | |
| | GGCGCTGTAGGTGTGGGGAAGAGTGCCCTGA | |
| | CCATCCAGCTTATCCAGAACCACTTTGTGGA | |
| | TGAGTACGACCCCACCATAGAGGTGAGCCTC | |
| | CAGCCCGCATCCCG | |
| Bovine | GGCTCCCTCGGCAGGTGGGACCGGCGACCAC | 45 |
| | TGCAGCGCCTCGTGCTGCGGTCTCTTGAGGA | |
| | GCAATGACGGAGTATAAGCTCGTGGTGGTGG | |
| | GCGCCGTTGGCGTGGGGAAGAGCGCCCTGAC | |
| | TATCCAGCTCATTCAGAATCACTTCGTGGAC | |
| | GAGTACGACCCCACCATCGAGGTGAGCCCGC | |
| | CTGGCTCCTGTCCG | |

Editing efficiencies in cell pools were verified at 2-3 time points post editing to screen for enrichment or depletion of desired mutations. Enrichment of a mutation of interest suggest a positive impact of a given mutation on cell growth, while depletion suggests a detrimental effect on cell health or growth. Editing efficiencies were measured by POR amplification of target region, Sanger Sequencing (Source Biosciences) and ICE analysis of the Sanger Sequencing file (Synthego).

### Growth Assays:

Cells were grown in adherence for multiple passages to obtain doubling time data and/or cumulative generation numbers. Cells were seeded at 2000-4000 cells/cm² into flasks coated with Matrigel or straight onto plastic (as annotated in graphs). Cells were counted and passaged every 3-4 days. Media for myoblasts was DMEM 4.5 g/L glucose, 20% FBS, 2 mM L-glutamine, 5 ng/μl FGF2. Media for ADSC was DMEM 1 g/L glucose, 10% FBS, 2 mM L-glutamine, 5 ng/μl FGF.

### Results

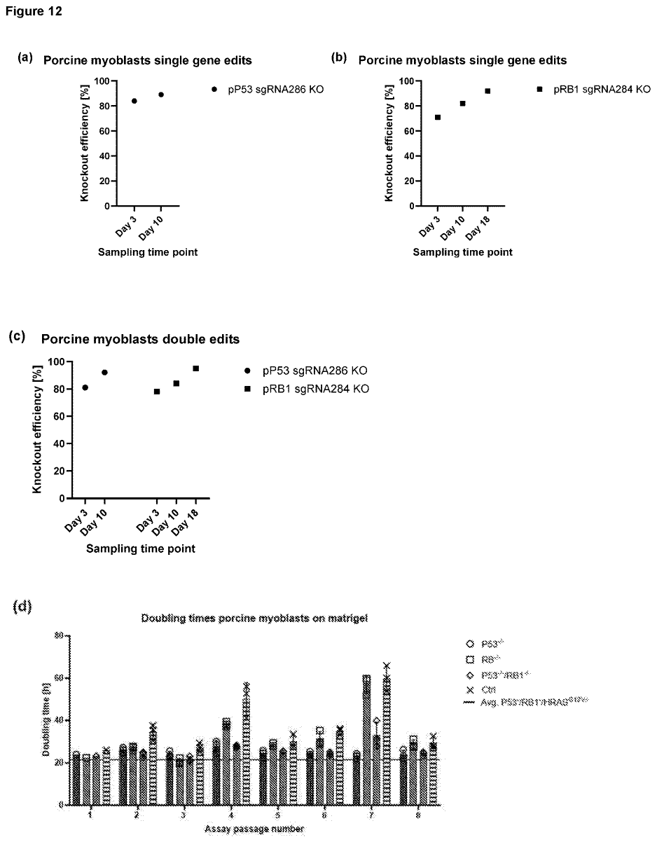

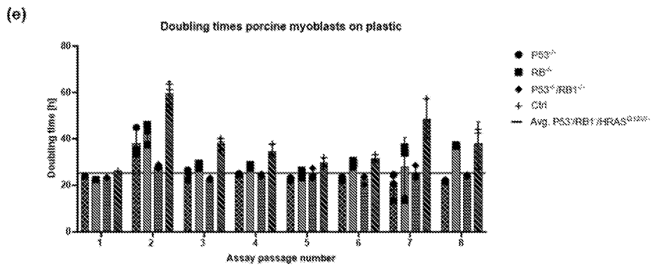

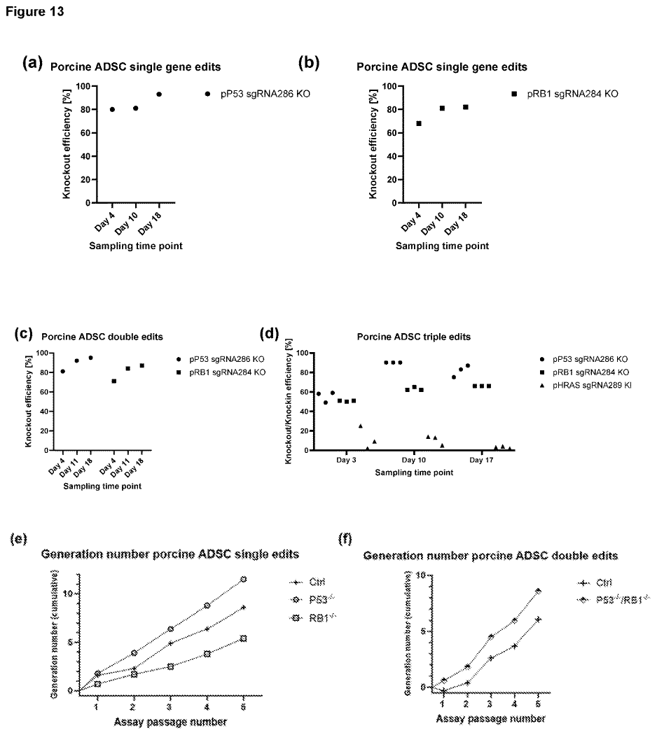

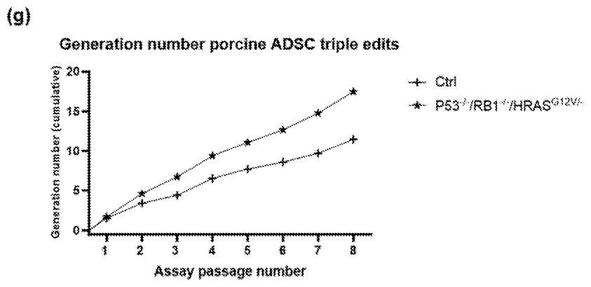

Cells were edited using one sgRNA against P53 (FIG. 12, panel a) and RB1 (FIG. 12, panel b) respectively or both in combination (FIG. 12, panel c). Editing efficiencies (knockout) were measured on two to three time points post editing. In all cases, P53 and RB1 edits increase or stay high over this period, suggesting a beneficial effect of those mutations on cell doubling times.

For a growth assay, cells were seeded into triplicate flasks (from growth period 2 onwards) and grown on Matrigel coated flasks (FIG. 12, panel d) or on plastic (FIG. 12, panel e) in adherence for 8 passages. Doubling times were compared to a control line (transfection with Cas9 only, no sgRNA; Ctrl). Amongst the single gene edited lines, the P53 knockout shows a growth advantage compared to the control while the RB1 knockout on its own does not seem to be sufficient to boost growth consistently in this cell line. The double edited P53^{-/-}/RB1^{-/-} line shows a growth advantage as well compared to the control lines in both conditions. As a reference point, the 8-passage average doubling time of a P53^{-/-}/RB1^{-/-}/HRAS^{G12V/-} cell line is plotted in the graphs shown in FIG. 12, panel d and FIG. 12, panel e as well. The P53^{-/-} single knockout line and the P53^{-/-}/RB1^{-/-} double knockout line show comparable doubling times to the P53^{-/-}/RB1^{-/-}/HRAS^{G12V/-} triple edited line in both conditions.

### Example 6: Editing Efficiencies and Growth Data from Porcine Adipose Derived Stem Cells (ADSC) Cell Lines

Cells were edited using one sgRNA against P53 (FIG. 13, panel a) and RB1 (FIG. 13, panel b) respectively or both in combination (FIG. 13, panel c) or as a triple edit together with an HRAS G12V knockin (FIG. 13, panel d, triplicate flasks). Editing efficiencies (knockout for P53 and RB1; knockin for HRAS) were measured on three time points post editing. In all cases, P53 and RB1 edits increase or stay high over this period, suggesting a beneficial effect of those mutations on cell doubling times. HRAS editing efficiencies are low and do not increase.

For a growth assay, cells were seeded into triplicate flasks and grown on plastic in adherence for 5-8 passages. Generation number accumulation was compared to a control line (transfection with Cas9 only, no sgRNA; Ctrl). Amongst the single gene edited lines (FIG. 13, panel e), the P53^{-/-} single knockout shows a growth advantage compared to the control while the RB1^{-/-} single knockout on its own does not seem to be sufficient to boost growth in this cell line. The double edited P53^{-/-}/RB1^{-/-} line (FIG. 13, panel f as well as the triple edited P53^{-/-}/RB1^{-/-}/HRAS^{G12V/-} (FIG. 13, panel g) both show growth advantages compared to their control lines.

### Example 7: Editing Efficiencies and Growth Data from Bovine Var. Angus Myoblast Cell Lines

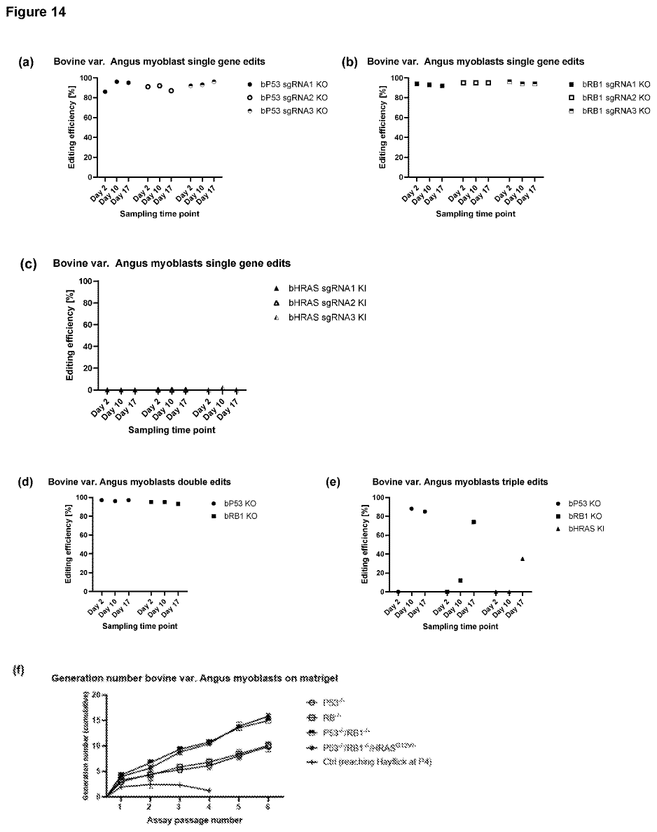

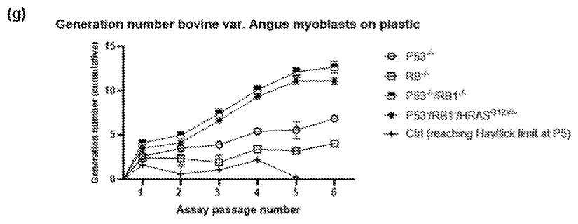

Cells were edited using one of three sgRNAs against bovine P53 (FIG. 14, panel a), RB1 (FIG. 14, panel b) or HRAS (FIG. 14, panel c) respectively or using bP53 sgRNA1 and bRB1 sgRNA1 in combination (FIG. 14, panel d) or as a triple edit using bP53 sgRNA3/bRB1 sgRNA3/HRAS sgRNA3 (FIG. 14, panel e). Editing efficiencies (knockout for P53 and RB1; knockin for HRAS) were measured on three time points post editing. In all cases, P53 and RB1 edits increase or stay high over this period, suggesting a beneficial effect of those mutations on cell doubling times. HRAS editing efficiencies are low and do not increase over time except in the last sampling time point.

For a growth assay, cells were seeded into triplicate flasks and grown on Matrigel (FIG. 14, panel f) or plastic (FIG. 14, panel g) in adherence for 6 passages where possible. Generation number accumulation was compared to a control line (transfection with Cas9 only, no sgRNA; Ctrl). All edited lines show growth advantages in both conditions over the control line which reached their Hayflick limit and died during the course of the growth assay. The best growth was observed in the P53^{-/-}/RB1^{-/-} double edited and P53^{-/-}/RB1^{-/-}/HRAS^{G12V/-} triple edited cell lines.

### Example 8: Editing Efficiencies from Bovine Var. Angus Adipose Derived Stem Cells (ADSC) Cell Line

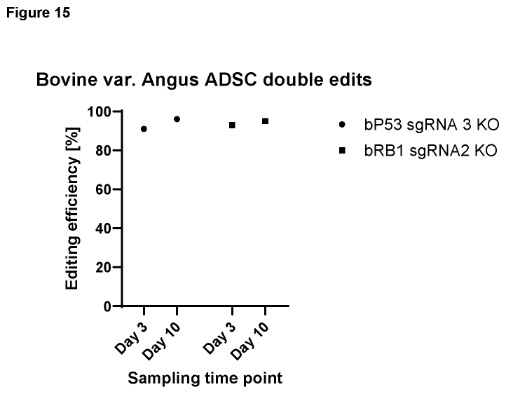

Cells were edited using bP53 sgRNA3 and bRB1 sgRNA2 in combination (FIG. 15). Editing efficiencies (knockout) were measured on two time points post editing. In all cases, P53 and RB1 edits increase or stay high over this period, suggesting a beneficial effect of those mutations on cell doubling times.

### Example 9: Editing Efficiencies and Growth Data from Bovine Var. Wagyu Myoblast Cell Lines

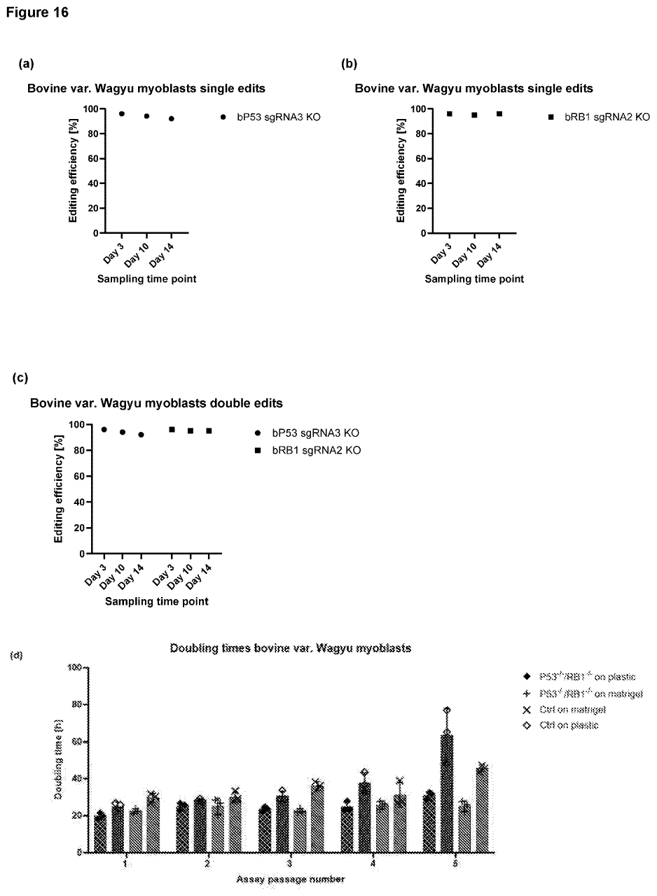

Cells were edited using one sgRNA against P53 (FIG. 16, panel a) and RB1 (FIG. 16, panel b) respectively or both in combination (FIG. 16, panel c). Editing efficiencies (knockout) were measured on three time points post editing. In all cases, P53 and RB1 edits stay high over this period, suggesting a beneficial effect of those mutations on cell doubling times.

For a growth assay, cells were seeded into triplicate flasks and grown on Matrigel coated flasks or on plastic (FIG. 16, panel d) in adherence for 5 passages. Doubling times were compared to a control line (transfection with Cas9 only, no sgRNA; Ctrl). The double edited P53^{-/-}RB1^{-/-} line shows a consistent growth advantage compared to the control lines in both conditions.

### Example 10: Editing Efficiencies and Growth Data from Bovine Var. Wagyu Adipose Derived Stem Cells (ADSC) Cell Lines

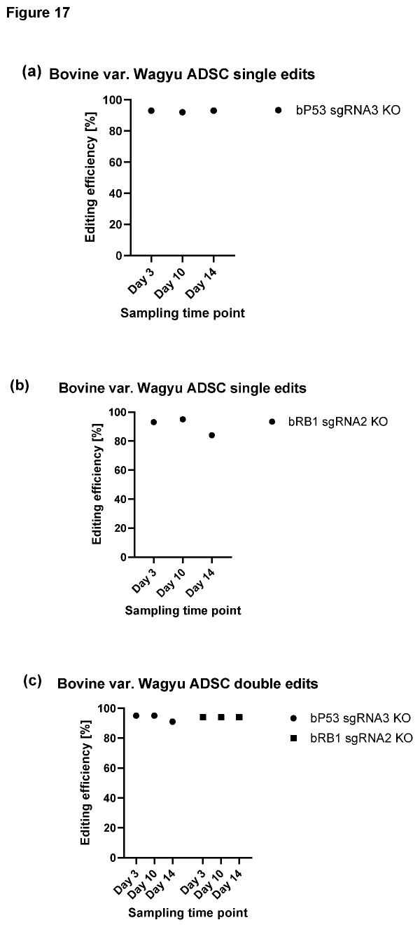

Cells were edited using one sgRNA against P53 (FIG. 17, panel a) and RB1 (FIG. 17, panel b) respectively or both in combination (FIG. 17, panel c). Editing efficiencies (knockout) were measured on three time points post editing. In all cases, P53 and RB1 edits stay high (>80%) over this period, suggesting a beneficial effect of those mutations on cell doubling times.

### Example 11: Editing Efficiencies from Chicken Myoblast Cell Lines

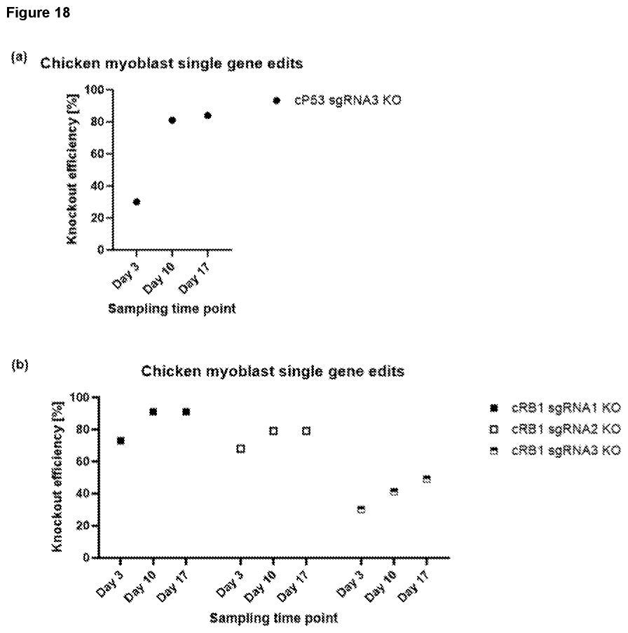

Cells were edited using one sgRNA against P53 (FIG. 18, panel a) or one out of three sgRNAs against RB1 (FIG. 18, panel b). Editing efficiencies (knockout) were measured on three time points post editing. In all cases, P53 and RB1 edits increase or stay high over this period, suggesting a beneficial effect of those mutations on cell doubling times.

TABLE 7
Sequences (pig)

| Description | SEQ ID NO. | Sequence |
|---|---|---|
| H-Ras - Wild Type (unedited). | SEQ ID NO. 1 | TCCTGTGCTGTGGCCTCCTGAGGA |
| Start codon | | GCGATGACGGAGTATAAGCTGGTG |
| is shown in bold. | | GTGGTGGGCGCTGGAGGTGTGGGG |
| | | AAGAGTGCCCTGACCATCCAGCTTA |
| | | TCCAGAACCACTTTGTGGATGAGTA |
| | | CGACCCCACCATAGAG |
| H-Ras - Knock-In of Donor 1. Gly > Val (aa12) | SEQ ID NO. 2 | TCCTGTGCTGTGGCCTCCTGAGGAG |
| [GGA > GTA] is shown in italics. PAM- | | CGATGACGGAGTATAAGCTGGTGG |
| blocking mutation Gly > Val (aa15) [GGG > | | TGGTGGGCGCTGtAGGTGTGGtGAAG |
| GtG] to prevent re-editing is shown in | | AGTGCCCTGACCATCCAGCTTATCC |
| underlined. Start codon is shown in bold and | | AGAACCACTTTGTGGATGAGTACGA |
| underlined. | | CCCCACCATAGAG |
| H-Ras - Knock-In of Donor 2. Gly > Val (aa12) | SEQ ID NO. 3 | TCCTGTGCTGTGGCCTCCTGAGGAGC |
| [GGA > GTA] is shown in italics. PAM- | | GATGACGGAGTATAAGCTGGTGGTGG |
| blocking mutation Gly > Leu (aa15) [GGG > | | TGGGCGCTGtAGGTGTGttGAAGAGT |
| Gtt] to prevent re-editing is shown in underlined. | | GCCCTGACCATCCAGCTTATCCAGAA |
| Start codon is shown in bold and underlined. | | CCACTTTGTGGATGAGTACGACCCCA |
| | | CCATAGAG |
| RB1 - Exon 8 - Wild Type (Unedited) | SEQ ID NO. 4 | TCCTGTGCTGTGGCCTCCTGAGGA |
| | | GCGATGACGGAGTATAAGCTGGTG |
| | | GTGGTGGGCGCTGGAGGTGTGGGG |
| | | AAGAGTGCCCTGACCATCCAGCTTA |
| | | TCCAGAACCACTTTGTGGATGAGTA |
| | | CGACCCCACCATAGAG |
| RB1 - Exon 8 - Edited (Major Allele) - | SEQ ID NO. 5 | TCCTGTGCTGTGGCCTCCTGAGGAG |
| delGG | | CGATGACGGAGTATAAGCTGGTGG |
| | | TGGTGGGCGCTGtAGGTGTGGtGAAG |
| | | AGTGCCCTGACCATCCAGCTTATCC |
| | | AGAACCACTTTGTGGATGAGTACGA |
| | | CCCCACCATAGAG |
| RB1 - Exon 8 - Edited (Minor Allele 1) - | SEQ ID NO. 6 | TCCTGTGCTGTGGCCTCCTGAGGAGC |
| delCG | | GATGACGGAGTATAAGCTGGTGGTGG |
| | | TGGGCGCTGtAGGTGTGttGAAGAGTG |
| | | CCCTGACCATCCAGCTTATCCAGAACC |
| | | ACTTTGTGGATGAGTACGACCCCACC |
| | | ATAGAG |
| PTEN - Exon 2 - Wild Type (Unedited) | SEQ ID NO. 7 | TCCTGTGCTGTGGCCTCCTGAGGA |
| | | GCGATGACGGAGTATAAGCTGGTG |
| | | GTGGTGGGCGCTGGAGGTGTGGGG |
| | | AAGAGTGCCCTGACCATCCAGCTTA |
| | | TCCAGAACCACTTTGTGGATGAGTA |
| | | CGACCCCACCATAGAG |
| PTEN - Exon 2 - Edited (Major Allele) - | SEQ ID NO. 8 | TCCTGTGCTGTGGCCTCCTGAGGAG |
| insA | | CGATGACGGAGTATAAGCTGGTGG |
| | | TGGTGGGCGCTGtAGGTGTGGtGAAG |
| | | AGTGCCCTGACCATCCAGCTTATCC |
| | | AGAACCACTTTGTGGATGAGTACGA |
| | | CCCCACCATAGAG |
| PTEN - Exon 2 - Edited (Minor Allele 1) - | SEQ ID NO. 9 | TCCTGTGCTGTGGCCTCCTGAGGAGC |
| delTA | | GATGACGGAGTATAAGCTGGTGGTGG |
| | | TGGGCGCTGtAGGTGTGttGAAGAGTG |
| | | CCCTGACCATCCAGCTTATCCAGAACC |
| | | ACTTTGTGGATGAGTACGACCCCACC |
| | | ATAGAG |
| TP53 - Exon 5 - Wild Type (Unedited) | SEQ ID NO. 10 | TCCTGTGCTGTGGCCTCCTGAGGA |
| | | GCGATGACGGAGTATAAGCTGGTG |
| | | GTGGTGGGCGCTGGAGGTGTGGGG |
| | | AAGAGTGCCCTGACCATCCAGCTTA |
| | | TCCAGAACCACTTTGTGGATGAGTA |
| | | CGACCCCACCATAGAG |
| TP53 - Exon 5 - Edited (Major Allele) - | SEQ ID NO. 11 | TACTCCCCTGCCCTCAATAAGCTG |
| insT | | TTTTGCCAGCTGGCCAAGACCTGC |
| | | CCGGTGCAGCTGTGGGTCAGCTC |
| | | GCCACCCCCGCCITGGCACCCGT |
| | | GTCCGCGCCATGGCCATCTACA |
| | | AGAAGTCAGAGTACATGACCG |
| | | AGGTGGTGAGGCGCTGTCCCCA |
| | | CCATGAGCGCAGCTCTGACTATAG |
| | | CGATG |
| TP53 - Exon 5 - Edited (Minor Allele 1) - | SEQ ID NO. 12 | TACTCCCCTGCCCTCAATAAGCTG |
| delT | | TTTTGCCAGCTGGCCAAGACCTGC |
| | | CCGGTGCAGCTGTGGGTCAGCTCG |
| | | CCACCCCCGCC-GGCACCCGTGTC |
| | | CGCGCCATGGCCATCTACAAGAAG |
| | | TCAGAGTACATGACCGAGGTGGT |
| | | GAGGCGCTGTCCCCACCATGA |
| | | GCGCAGCTCTGACTATAGCGATG |
| H-RAS (Knock-Out) - Exon 2 - Wild Type | SEQ ID NO. 13 | TCCTGTGCTGTGGCCTCCTGAGGAG |
| (Unedited) | | CGATGACGGAGTATAAGCTGGTGG |
| | | TGGTGGGCGCTGGAGGTGTGGGG |
| | | AAGAGTGCCCTGACCATCCAGCTT |
| | | ATCCAGAACCACTTTGTGGATGAG |
| | | TACGACCCCACCATAGAG |
| H-RAS (Knock-Out) - Exon 2 - Edited | SEQ ID NO. 14 | TCCTGTGCTGTGGCCTCCTGAGGA |
| (Major Allele) - insT | | GCGATGACGGAGTATAAGCTGGT |
| | | GGTGGTGGGCGCTGGAGGITGTG |
| | | GGGAAGAGTGCCCTGACCATCCA |
| | | GCTTATCCAGAACCACTTTGTGG |
| | | ATGAGTACGACCCCACCATAGAG |

### REFERENCES

T Dull, R Zufferey, M Kelly, R J Mandel, M Nguyen, D Trono, L Naldini. A third-generation lentivirus vector with a conditional packaging system. J Virol. 1998 November; 72(11):8463-71. doi: 10.1128/JVI.72.11.8463-8471.1998.
Ran, F., Hsu, P., Wright, J. et al. Genome engineering using the CRISPR-Cas9 system. Nat Protoc 8, 2281-2308 (2013). https://doi.org/10.1038/nprot.2013.143

TABLE 8
Ras isoform amino acid sequences (pig)

| | | |
|---|---|---|
| H-Ras - Wild | SEQ ID | MTEYKLVVVGAGGVGKSALTIQLIQ |
| Type amino | NO. 46 | NHFVDEYDPTIEDSYRKQVVIDGET |
| acid sequence | | CLLDILDTAGQEEYSAMRDQYMRTG |
| (unedited). | | EGFLCVFAINNTKSFEDIHQYREQI |
| | | KRVKDSDDVPMVLVGNKCDLAARTV |
| | | ESRQAQDLARSYGIPYIETSAKTRQ |
| | | GVEDAFYTLVREIRQHKVRKLSPPD |
| | | EGGPGCLSCKCLLS |
| K-Ras - Wild | SEQ ID | MTEYKLVVVGAGGVGKSALTIQLIQ |
| Type amino | NO. 47 | NHFVDEYDPTIEDSYRKQVVIDGET |
| acid sequence | | CLLDILDTAGQEEYSAMRDQYMRTG |
| (unedited). | | EGFLCVFAINNTKSFEDIHHYREQI |
| | | KRVKDSEDVPMVLVGNKCDLPSRTV |
| | | DTKQAQDLARSYGIPFIETSAKTRQ |
| | | RVEDAFYTLVREIRQYRLKKISKEE |
| | | KTPGCVKIKKCIIM |
| N-Ras - Wild | SEQ ID | MTEYKLVWVGAGGVGKSALTIQLIQ |
| Type amino | NO. 48 | NHFVDEYDPTIEDSYRKQVVIDGET |
| acid sequence | | CLLDILDTAGQEEYSAMRDQYMRTG |
| (unedited). | | EGFLCVFAINNSKSFADINLYREQI |
| | | KRVKDSDDVPMVLVGNKCDLPTRTV |
| | | DTKQAHELAKSYGIPFIETSAKTRQ |
| | | GVEDAFYTLVREIRQYRMKKLNSSD |
| | | DGTQGCMGLPCVVM |

1. A modified animal cell having a genetic modification in one or more genes associated with genome surveillance, cell cycle control and/or cell death control, wherein the animal is of an animal species suitable for human or animal consumption, optionally wherein the animal is used in agriculture.

2. A modified animal cell according to claim 1, wherein the modification immortalises the cell.

3. A modified animal cell according to claim 1, wherein the animal is selected from a pig, bovine, poultry, sheep, goat, fish, crustaceans or mollusc.

4. The modified animal cell according to claim 1, wherein the modified cell is a somatic cell, optionally wherein the modified cell is selected from one of the following cell types: myoblast, fibroblast, myofibroblast, adipose derived stem cell, epithelial cell, mesenchymal stem cell, satellite cell or hepatocyte.

5. (canceled)

6. The modified animal cell according to claim 1, wherein said cell does not express an exogenous nucleic acid to manipulate the genome surveillance, cell cycle control and/or cell death control pathway.

7. The modified animal cell according to claim 1, wherein the animal cell has a genetic modification in one or more of the following genes: RB1, TP53, and/or a RAS gene.

8-14. (canceled)

15. The modified animal cell according to claim 7, wherein the RAS gene is HRAS, NRAS, or KRAS.

16. The modified animal cell according to claim 1, wherein the modification is in the promoter region or coding region of the one of more genes, and/or wherein the modification is introduced using targeted genome modification, optionally wherein the modification is introduced using an endonuclease, preferably wherein the endonuclease is selected from TALEN, ZFN or CRISPR/Cas9.

17-20. (canceled)

21. The modified animal cell according to claim 120, wherein the modification is a loss of function modification and comprises a knock-out of the gene.

22. The modified animal cell according to claim 1, wherein the gene is a RAS gene and the modification is a hyperactivation modification, optionally, wherein the RAS gene is HRAS, NRAS, or KRAS.

23. The modified animal cell according to claim 22, wherein the hyperactivation modification comprises one or more amino acid substitutions, optionally
wherein the one or more amino acid substitutions comprises substituting the glycine at positions 12 or 13 or the glutamine at position 61 of SEQ ID NO: 46, 47, or 48: or
wherein the one or more amino acid substitutions comprises substituting the glycine at positions 12 or 13 of SEQ ID NO: 46, 47, or 48, wherein the one or more amino acids is selected from a list comprising alanine, cysteine, aspartic acid, arginine, serine, and valine or wherein the one or more amino acid substitutions comprises substituting the glycine at positions 12 or 13 of SEQ ID NO: 46, 47, or 48 with valine; or
wherein the one or more amino acid substitutions comprises substituting the glycine at position 61 of SEQ ID NO: 46, 47, or 48, wherein the one or more amino acids is selected from a list comprising glutamic acid, histidine, lysine, proline, and arginine.

24-27. (canceled)

28. A method of producing cultivated meat or a cultured meat product comprising culturing the modified animal cell according to claim 1.

29. The method according to claim 28, wherein the method comprises continuous or batch culture of the modified cell and/or comprises the step of forming the cells into a tissue like structure, and/or wherein the cells are formed into a muscle tissue like structure.

30-31. (canceled)

32. A method of producing the modified animal cell according to claim 1, wherein the method comprises introducing a genetic modification in one or more genes associated with genome surveillance, cell cycle control and/or cell death control and wherein the animal is an animal suitable for human or animal consumption, optionally wherein the animal is used in agriculture.

33. (canceled)

34. The method according to claim 32, wherein the genetic modification alters the expression or function of one or more genes associated with genome surveillance, cell cycle control and/or cell death control.

35. The method according to claim 32, wherein the animal cell is immortalized.

36. The method according to claim 32, wherein the animal is selected from a pig, bovine, poultry, sheep, goat, fish, crustaceans or mollusc.

37. The method according to claim 32, wherein the modified cell is a somatic cell, optionally wherein the modified cell is selected from one of the following cell types: myoblast, fibroblast, myofibroblast, adipose derived stem cell, epithelial cell, mesenchymal stem cell, satellite cell or hepatocyte.

38. (canceled)

39. The method according to claim 32, wherein said cell does not express an exogenous nucleic acid to manipulate the genome surveillance, cell cycle control and/or cell death control.

40. The method according to claim 32, wherein the animal cell has a genetic modification in one or more of the following genes: RB1, TP53, and/or a RAS gene.

41-47. (canceled)

48. The method according to claim 40 wherein the RAS gene is HRAS, NRAS, or KRAS.

49-69. (canceled)
